(19) United States
(12) Patent Application Publication
(10) Pub. No.: US 2003/0065450 A1
(43) Pub. Date: Apr. 3, 2003

(54) Apparatus and method for providing hair tinting information

(76) Inventor: Soazig Leprince, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/098,574

(22) Filed: Mar. 18, 2002

(30) Foreign Application Priority Data
Mar. 18, 2001 (FR) 01 03602

(51) Int. Cl. G06F 19/00; G01N 33/48; G01N 33/50

(52) U.S. Cl. 702/19

(57) ABSTRACT

An apparatus provides hair tinting information. The apparatus may include a data processor and a memory functionally coupled to the data processor. In one embodiment, the memory stores information relating to a set hair tinting products and instructions to be executed by the data processor, for: receiving first information representative of a subject's percentage of senescent hair; receiving second information representative of the subject's natural hair color; determining, based on the first information, the second information, and information relating to a set of hair tinting products, types of attainable color results; and providing, based on the types of attainable color result and the set of hair tinting products, information relating to at least one hair tinting product. In addition to various forms of the apparatus, there are various methods of providing hair tinting information.

The present invention relates to an apparatus and method providing information relating to hair tinting.

To guide consumers in choosing tinting products, in particular choosing tinting creams and/or liquids for dying hair to a desired color regardless of its starting color, hair tinting products often include a color table on the packaging. The table may display three starting colors, namely fair hair, medium hair, and dark hair, together with three corresponding colors as obtained after applying the tinting product.

For products that have a lightening effect, hair tinting products provide tables of colors corresponding to hair color before and after application of the tinting product, together with the results obtained by means of other products in the range. This makes it easier to choose the range of color and the product which will obtain the lightness most suited to the natural color of the hair.

Selecting a tinting product turns out in practice to be quite difficult, since the consumer must sort through the large amount of information present on all of the packaging present on the shelves, and often may have only a limited amount of time available for doing this.

In addition, the information carried on the packaging may not always be very precise and can lead the consumer to believe that a particular color result can be obtained whereas in fact some neighboring color will be obtained, and that can lead to a disappointing result.

At the time of filing the French priority application relied on for the benefit of priority, there was an Internet site including a personalized diagnosis system enabling an individual to provide information concerning hair color and percentage of senescent hairs. The site determined whether products in various ranges offered by a company were suitable, given the natural color of the hair and the percentage of senescent hair. The set of ranges was displayed on the screen and the subject was able to select a range by clicking on it.

Whenever the subject's hair color or percentage of senescent hair was unsuitable, a message was displayed to indicate that a particular selected range may not be recommended.

Otherwise, the various tinting shades that could be obtained using the products in the selected range were displayed on the screen, and by clicking on a particular shade, information about the corresponding tinting product was displayed, wherein the displayed information was a reproduction of the table of shades printed on the packaging of the product.

In some circumstances, such a site may not provide full satisfaction. For example, it may be of interest to provide a consumer with advice about where the products are sold. In addition, it may be of interest to avoid having the consumer select from each range of products in order to discover whether the products in the range are suitable, which can be tiresome and time consuming when the number of ranges is large.

As embodied and broadly described herein, the invention may comprise an apparatus for providing hair tinting information which may include, a data processor, and a memory functionally coupled to the data processor, wherein the memory stores information relating to a set hair tinting products and instructions to be executed by the data processor, for: receiving first information representative of a subject's percentage of senescent hair; receiving second information representative of the subject's natural hair color; determining, based on the first information, the second information, and information relating to a set of hair tinting products, attainable types of color result; and providing, based on the attainable types of color result and the set of hair tinting products, information relating to at least one hair tinting product.

In another aspect, the apparatus may comprise a display functionally coupled to the data processor.

In yet another aspect, the apparatus may include an input device functionally coupled to the data processor, wherein the input device is at least one of a keyboard, a mouse, and a measuring device, wherein the measuring device may comprise a portable instrument for measuring a subject's hair color.

In a further aspect, the display may include a touch screen display configured to receive input commands.

In yet another aspect the apparatus may further comprise a network interface functionally coupled to the data processor and a network.

In still another aspect, the apparatus may comprise a support structure which supports the data processor and the display.

In further aspect, the apparatus may include instructions stored in memory, to be executed by the data processor, for receiving information representative of a desired color result chosen from the attainable types of color result, and wherein the providing is further based on the information representative of a desired color result.

In another aspect, the apparatus may include information relating to at least one hair tinting product, wherein the information may comprise information relating to ranges of hair tinting products, and wherein the apparatus may further comprise additional instructions stored in memory executed by the data processor, for receiving a subject's selection of at least one desired range, wherein the desired range provides shades attainable through one of direct application (of one or more products) and (application of one or more products after) a prior application of a bleach.

In another aspect, the apparatus may comprise additional instructions to be executed by the data processor for providing, based on the natural hair color, percentage of senescent hair, and the information relating to the set of hair tinting products, information relating to at least one duration, wherein the at least one duration corresponds to a temporal duration of the color result, and receiving third information representative of a desired duration chosen from the at least one duration.

In yet another aspect, the receiving third information representative of a desired duration may include receiving one of a confirmation of a desired duration and a selection of one desired duration from a plurality of durations.

In another aspect, the apparatus may comprise additional instructions to be executed by the data processor for receiving data representative of the set of hair tinting products, wherein the data may include information representative of the ability to cover senescent hairs and the attainable types of color result.

In still another aspect, the data representative of the set of hair tinting products may include information representative of a product duration, and at least one of a natural application base, a lightening power, and a bleaching power.

In another aspect, the apparatus may further comprise additional instructions to be executed by the data processor for receiving information representative of a subject's complexion, wherein the providing is further based on the information representative of the subject's complexion.

In yet another aspect, the apparatus may further comprise additional instructions to be executed by the data processor for selecting, from the set of hair tinting products, a first subset of hair tinting products corresponding to the first information, wherein the selecting is performed using a first filter.

In another aspect, the apparatus may further comprise additional instructions to be executed by the data processor for selecting, from the first subset of hair tinting products, a second subset of hair tinting products corresponding to the second information, wherein the selecting is performed using a second filter.

In another aspect, the first filter selects hair tinting products providing appropriate coverage based on the subject's percentage of senescent hair.

In still another aspect, for each determined type of attainable type of color result offered, the second filter selects, from the first subset, at least one of: a) hair tinting products having a natural application base corresponding to the second information, and b) hair tinting products satisfying a condition specific to types of color results.

In another aspect, the apparatus may further comprise providing an attainable type of color result corresponding to blonde hair for the whole head when a condition C+Pe+Pd≧8 is satisfied, wherein C represents the subject's natural hair color, Pe represents a lightening power, and Pd represents a bleaching power.

In another aspect, the apparatus may further comprise additional instructions for execution on the data processor for providing an attainable type of color result corresponding to blonde hair over only a portion of the hair when the hair tinting product is suitable for application to hair having the same color as the subject's natural hair color.

In still another aspect, the apparatus may further comprise additional instructions for execution on the data processor for providing an attainable type of color result corresponding to hair that is not blonde and having a color that is different from the at least a portion of the subject's natural hair color when the hair tinting product is suitable for application to hair having the same color as the subject's natural hair color.

In yet another aspect, the apparatus may further comprise providing an attainable type of color result corresponding to shading senescent hair, when the hair tinting product is suitable for application to hair having the same color as the subject's natural hair color.

In another aspect, the second subset of hair tinting products may comprise at least one duration, and wherein the method may comprise providing the at least one duration.

In still another aspect, the apparatus may further comprise additional instructions for execution on the data processor for selecting, from the second subset of hair tinting products, a third subset of hair tinting products corresponding to a desired duration, wherein the selecting is performed using a third filter.

In another aspect, wherein the set of hair tinting products may include ranges of hair tinting products, and wherein each range may include at least one hair tinting product.

In still another aspect, the apparatus may further comprise additional instructions for execution on the data processor for reading, from at least one of the second subset and the third subset, ranges associated with the hair tinting products.

In another aspect, the apparatus may further comprise additional instructions for execution on the data processor for providing shades of the hair tinting products corresponding to a desired range selected by the subject.

In still another aspect, the apparatus may further comprise additional instructions for execution on the data processor for reading, from at least one of the second subset and the third subset, at least one shade associated with the at least one hair tinting product, wherein the shade is suitable for the subject's complexion.

In another aspect, the apparatus may further comprise an image acquisition device for capturing an image of the subject for displaying on the display.

In another aspect, the image acquisition device may comprise a camera.

In yet another aspect, the apparatus may further comprise additional instructions for execution on the data processor for overlaying a second image on a portion of the subject's image corresponding to the subject's hair, wherein the second image may be configured for a) comparing the color of the subject's hair with the at least one shade and, b) comparing the subject's percentage of senescent hair with at least one example of percentage of senescent hair.

In another aspect, the apparatus may further comprise additional instructions for execution on the data processor for enabling the subject to purchase at least some of the hair tinting products.

In another aspect, the apparatus may further comprise additional instructions for execution on the data processor for offering at least some of the hair tinting products for sale.

In yet another aspect, the apparatus operates at least substantially entirely at a point of sale for the hair tinting products.

In still another aspect, the information relating to at least one hair tinting product may comprise information relating to at least one of a plurality of products and a plurality of subsets of products.

In yet another aspect, the first information and second information may be received via an input device and the providing may comprise displaying the information relating to at least one product on a display device.

In another aspect, at least the receiving of the second information may comprise receiving information obtained via a measurement device.

In a further aspect, at least one of the receiving of first information, the receiving of second information, and the providing may occur via a network.

In yet another aspect, the apparatus may further comprise additional instructions for execution on the data processor for prompting the subject to compare the subject's hair with one of a plurality of color swatches representing natural hair color and a plurality of color swatches representing percentages of senescent hair.

In another aspect, the apparatus may operate substantially as a stand-alone unit without being connected to a computer server.

In still another aspect, the apparatus may further comprise a mirror.

In another aspect, the apparatus may further comprise at least one swatch sampler.

In another aspect, the at least one swatch sampler may include a sampler of colored swatches comprising shade colors of black, brown, dark chestnut, chestnut, pale chestnut, dark blonde, blonde, light blonde, very light blonde, and lightest blonde.

In a further aspect, the apparatus may further comprise at least one swatch sampler for estimating the quantity of senescent hair.

In another aspect, the at least one swatch sampler may further include two sets of samplers enabling the quantity of senescent hair to be evaluated, one corresponding to a subject having fair hair and one corresponding to a subject having dark hair.

In yet another aspect, the at least one swatch sampler for estimating the quantity of senescent hair may be comprised of at least five swatches, corresponding to senescent hair percentages of 0%, less than 30%, 30% to 50%, 50% to 80%, and greater than 80%.

In another aspect, the apparatus may be adjacent to at least one set of shelves suitable for containing hair tinting products.

In still another aspect, the apparatus may further comprising a lighting means for lighting a subject's hair.

The term “senescent hair” refers to hair having color generally associated with aging. Senescent hair may refer to white hair, gray hair, or any perceived and/or measured color associated with the aging process of human hair.

A “type of color result” refers generally to information describing a hair tinting effect. A type of color result may be broader than a final hair color a subject wishes to obtain. It can be a word or expression generally describing a color result and/or indicating if hair color is substantially modified after tinting, and/or if tinting only applies to strands or to the whole head. It may also express a purpose for the hair coloration, such as, for example, covering senescent hair.

A “hair tinting result” generally refers to a type of color result and the duration associated therewith.

“Receiving” may include at least one of acquisition via a network, via a measurement device, via verbally communication, via electronic transmission, via telephone transmission, interactive voice response system, in hard-copy form, or through any other mechanism enabling reception. Receiving may include accessing and/or storing data from/to a database, data structure, storage medium, survey, and/or any other mechanism or combination of mechanisms. The received data may be raw data, such as data entries from a database, preprocessed data, such as encoded raw data, or any other form of data. In addition, “receiving” may occur either directly or indirectly. For example, receipt may occur through a third entity acting in concert with another. Regardless, all such indirect and direct actions are intended to be covered by the term “receiving” as used herein. Received information, for example, may take one of many forms. It may simply be a checked box, clicked button, a result of a touch screen selection, or data from a measurement device. Receiving may occur through an on-line site, e-mail, facsimile, telephone, interactive voice response system, or file transfer protocol transmitted electronically over a network at a web site, an internet protocol address, or a network account. Information may be received from a subject for whom information is sought, or an entity acting on the subject's behalf. “Receiving” may involve receipt directly or indirectly through one or more networks and/or storage mediums. Examples of storage media may include magnetic storage devices such as floppy disks and hard drives, optical storage devices, such as compact discs and digital video discs, organic storage devices, solid-state memory, printed media, and/or any other medium for storing information.

As used herein, the term “providing” may refer to making available for use and/or display, giving, generating for use, and/or producing. Providing may be performed manually or in an automated fashion. “Providing” may be performed physically and/or electronically, which may include at least one of producing via a network, via verbal communication, via electronic transmission, via telephone transmission, interactive voice response system, in hard-copy form, or through any other mechanism enabling reception. “Providing” may include accessing and/or storing data from/to a database, data structure, storage medium, survey, and/or any other mechanism or combination of mechanisms. The provided data may be raw data, such as data entries from a database, preprocessed data, such as encoded raw data, or any other form of data. In addition, “providing” may occur either directly or indirectly. For example, providing may occur through a third entity acting in concert with another. Regardless, all such indirect and direct actions are intended to be covered by the term “providing” as used herein. Providing may occur through an on-line site, e-mail, facsimile, telephone, interactive voice response system, or file transfer protocol transmitted electronically over a network at a web site, an internet protocol address, or a network account. “Providing” may involve an exchange of information directly or indirectly through one or more networks, output devices and/or storage mediums. Examples of output devices may include Cathode Ray Tube or Liquid Crystal Displays, touch-screen displays, numerical digital displays, and/or hardcopy output.

The term “network” may include a public network such as the Internet or a telephony network, a private network, a virtual private network, or any other mechanism for enabling communication between two or more nodes or locations. The network may include one or more of wired and wireless connections. Wireless communications may include radio transmission via the airwaves, however, those of ordinary skill in the art will appreciate that various other communication techniques can be used to provide wireless transmission including infrared line of sight, cellular, microwave, satellite, blue-tooth packet radio and spread spectrum radio. Wireless data may include, but is not limited to, paging, text messaging, e-mail, Internet access and other specialized data applications specifically excluding, or including voice transmission.

In some instances consistent with the invention, a network may include a courier network (e.g. postal service, United Parcel Service, Federal Express, etc.). Other types of networks that are to be considered within the scope of the invention include local area networks, metropolitan area networks, wide area networks, ad hoc networks, or any mechanism for facilitating communication between two nodes or remote locations.

An “input device” is defined broadly to include any device capable of acquiring information. Input devices may include, singly or in combination, a keyboard, a touch screen display, a mouse, or any other device capable of acquiring information. Input devices may also include a portable, hand-held apparatus, which may, at least in part, automatically acquire information.

A further aspect relates to an apparatus for providing personalized advice concerning hair tinting, in particular at a point-of-sale. Such an apparatus may comprise a display device, a data processor apparatus suitable for controlling the display device in particular to display various messages and/or images thereon, and an input device enabling input of information into the data processor apparatus. The input device may enable the input of information concerning natural hair color of the subject and the subject's percentage of senescent hair. The data processor may have access to stored data relating to the tinting characteristics of a set of listed products that can be offered to the subject, and the data processor apparatus may be arranged to determine, on the basis of the data related to the subject and of the stored data, the types of color result that can be attained, and propose products or sets of products listed as a function of the types of color result that can be attained and the type of color result that is desired.

In at least one exemplary embodiment of the invention, selection of a hair tinting product may be simplified since the subject does not need to consult all of the packages of the various tinting products offered for sale.

The subject may be informed directly, as a function of the desired type of color result, to consider only products relevant to the desired type of color result, thus saving time.

In yet another aspect, the data processor apparatus may be arranged firstly, to ask the subject for the subject's natural hair color and quantity of senescent hair, secondly, to respond to the data from the subject to determine the types of color result that can be obtained given the characteristics of the listed products, thirdly, to ask the subject for the desired type of color result from amongst those which can be attained, fourthly, having regard to the characteristics of the listed products, to propose ranges of products that enable the desired type of color result to be obtained; and finally, to ask the subject to select one or more ranges from amongst those proposed, and in each selected range to indicate the shades that are directly attainable or that are attainable after prior application of a bleach.

In still another aspect, the apparatus may arranged to offer the subject one or more durations concerning how long the result will last as a function of the natural color of the subject's hair, the quantity of senescent hair, and the characteristics of the listed products, and to enable the subject to confirm the duration when there is only one duration or to select amongst a plurality of durations when there are at least two.

In an exemplary embodiment where the apparatus takes account of the length of time the subject would like the result to last, selection of the product may be facilitated, making it possible in particular to reduce the time for selecting a product. Such reduction of time could be of interest at a point-of-sale where there are a great number of consumers.

In a further aspect, for each listed product, the stored data may comprise at least: covering ability concerning senescent hairs, and the type of color result that the product makes it possible to obtain.

In another aspect, for each listed product, the stored data may further comprise the length of time the result lasts, and the natural application bases and/or the lightening or bleaching power of the product.

The data processor apparatus may be arranged to request the subject's complexion and to select amongst the listed products those which are listed as suiting the complexion specified. Thus, the apparatus makes it possible to match the color of the tinting with that of the subject's complexion.

In a further aspect, the data processor apparatus may include a first filter for selecting amongst the listed products those which are applicable for a given quantity of senescent hairs.

In another aspect, the data processor apparatus may include a second filter for selecting for each type of color result that can be offered, those in which the natural color of the hair and the natural application bases of the listed products are compatible, when this information is available in the stored data, and/or a condition specific to the type of color result that can be offered is satisfied by the natural color of the hair and by the lightening or bleaching power of the products.

In another aspect, the apparatus can be arranged, after applying the first and second filters, to offer a first type of color result corresponding to blonde hair for the whole head whenever the condition C+PE+PD≧8 is satisfied, where C is the natural color of the hair, C being expressed as a shade number lying in the range 1 to 10 from darkest to fairest, P_{E} is the lightening power expressed as a difference in shade numbers, and P_{D} is the bleaching power expressed as a difference of shade numbers when it is possible to use a bleach, and/or the stored data associated with each product may include an indication as to whether the product is suitable for application to hair having the same color as the subject's hair.

The data processor apparatus can also be arranged, after applying the first and second filters, to offer a second type of color result corresponding to blonde hair over a portion only of the hair, whenever the data stored in association with each listed product may include an indication as to whether the product is suitable for application to hair having the color of the subject's hair.

The data processor apparatus can also be arranged, after applying the first and second filters, to propose a third type of color result corresponding to hair that is not blonde and having a color that is different from its natural color over all or part of the hair, whenever the data stored in association with each listed product may include an indication as to whether the product is suitable for application to hair having the color of the subject's hair.

The data processor apparatus can also be arranged, after applying the first and second filters, to offer a fourth type of color result corresponding to shading senescent hair, whenever the data stored in association with each listed product may include an indication as to whether the product is suitable for application to hair having the color of the subject's hair.

The data processor apparatus may include a first read engine for responding to the products selected by applying the first and second filters, by reading at least one duration during which the result will last and by causing the result durations that appear amongst the products selected in this way to be displayed.

Optionally, the apparatus may include a third filter for selecting from amongst the products selected by applying the first and second filters, those products which have durations over which the result lasts that correspond to the duration selected by the subject, said duration being selected from amongst those determined by the first read engine.

The data stored for each listed product may include an indication concerning a determined range of products to which the product belongs.

In still another aspect, the data processor apparatus may include a second read engine for reading the ranges of products to which the selected products belong after applying at least the first and second filters, and preferably also after applying the third filter.

In another aspect, the apparatus may include a shade display engine for displaying the shades of the products in a range of products selected by the subject from amongst the ranges of products determined by the second read engine.

Optionally, the apparatus may further include a third read engine for reading the shades suitable for a complexion of a given color from amongst the products selected by applying the first and second filters and preferably also the third filter, for determining those products which are listed as being more particularly suitable for the complexion color given by the subject.

Optionally, the apparatus may be connected to a network, in particular, via the Internet, to a remote server for communications using the IP protocol.

In yet another aspect, the apparatus may include a mirror for enabling a subject to compare the color of their hair more easily with that of a palette, and also making it easier to determine the percentage of senescent hairs.

Optionally the apparatus may comprise an image acquisition element, such as a camera.

In a variant, the mirror may be replaced by an image acquisition element, such as a camera for picking up an image of the subject's hair, and an image insertion engine enabling the displayed image to have overlaid thereon an image of the subject's hair so as to make it easier for the subject to compare the color of the subject's own hair or the quantity of senescent hairs therein with the examples displayed on the screen for comparison purposes or with swatches in a swatch sampler.

Optionally, the apparatus may also have both a mirror and a camera.

In still another aspect, the display device may comprise a touch-sensitive screen.

In another aspect, a method for providing hair tinting information may comprise receiving first information representative of at least one state of a subject's hair, receiving second information representative of at least one desired hair tinting result of the subject, providing information relating to a plurality of hair tinting products based on at least the first information and the second information, receiving third information representative of the subject's selection of at least one hair tinting product in the plurality of hair tinting products, and providing information relating to the at least one selected hair tinting product.

In another aspect, the receiving first information may include receiving at least one of a subject's natural hair color and a subject's percentage of senescent hair.

In another aspect, the first information may be received from at least one of a portable measuring device, a touch-screen display, a mouse, and a keyboard.

In yet another aspect, the method may further comprise, providing a plurality of natural hair color examples, wherein receiving first information may comprise receiving a subject's natural hair color selection corresponding to one of the plurality of natural hair color examples which best corresponds to the subject's natural hair color, and storing the subject's natural hair color selection.

In yet another aspect, the method may further comprise providing a plurality of examples of senescent hair percentages, wherein the receiving first information may comprise receiving a subject's percentage of senescent hair selection corresponding to one of the plurality of examples of senescent hair percentages which best corresponds to the subject's percentage of senescent hair.

In yet another aspect, the method may further comprise receiving product information corresponding to the plurality of hair tinting products, wherein the received product information may include at least one of suitability of covering a percentage of senescent hair, natural application base, color effect obtained, color trademark, base shade, shade of highlights, lightening power, bleaching power, tone, product range, and product duration.

In another aspect, the product information may be received from a local storage medium, including at least one of a hard disk and a solid state memory.

In yet another aspect, the product information may be received over a network.

In yet another aspect, the network may utilize at least internet protocol.

In yet another aspect, the product information may be representative of products marketed under at least one brand name.

In yet another aspect, the method may further comprises selecting from the plurality of hair tinting products, based on the subject's percentage of senescent hair, a first subset of hair tinting products, and selecting from the first subset, based on the subject's natural hair color, a second subset of hair tinting products.

In yet another aspect, the selecting from the first subset may comprise selecting at least one of a) hair tinting products having a natural application base corresponding to the subject's natural hair color, and b) hair tinting products which satisfy at least one condition specific to at least one type of color result.

In yet another aspect, the receiving second information may include receiving a desired type of color result by the user, wherein the desired type of color result is selected from attainable types of color result.

In yet another aspect, the attainable types of color result may include at least one of lightness, colored shade, and cover senescent hair.

In yet another aspect, the lightness may include at least one of streaks and whole head.

In yet another aspect, the colored shade may include at least one of intense result, natural result, and colored streaks result.

In yet another aspect, the receiving second information may include receiving the desired duration of hair tinting.

In still another aspect, the method may further comprise determining the product durations for at least some of the hair tinting products, and selecting hair tinting products having a product duration corresponding to the desired duration.

In yet another aspect, the providing information relating to a plurality of hair tinting products may further comprise providing ranges of hair tinting products.

In yet another aspect, the receiving third information representative of the subject's selection may further comprise receiving information associated with at least one desired range.

In yet another aspect, the method may further comprise receiving a selection of at least one desired range of hair tinting products, and providing information relating to the hair tinting products having at least one range corresponding to the at least one desired range.

In yet another aspect, at least one shade may be associated with the at least one range.

In yet another aspect, the method may further comprise receiving information related to a subject's desired tone, determining the hair tinting products within the at least one range having a tone corresponding to the desired tone, and indicating at least one shade which corresponds to the desired tone.

In yet another aspect, the method may further comprise enabling the subject to purchase at least some of the hair tinting products.

In yet another aspect, the method may further comprise offering at least some of the hair tinting products for sale.

In yet another aspect, the method may be performed at least substantially entirely at a point of sale for the hair tinting products.

In yet another aspect, the information relating to at least one hair tinting product may comprise information relating to at least one of a plurality of products and a plurality of subsets of products.

In still another aspect, a method for providing hair tinting information may comprise receiving first information representative of at least a desired type of color result of a subject, providing at least one type of duration based at least on the first information, receiving second information representative of a duration chosen from the at least one type of duration, providing, based on at least the first information and the second information, information relating to a plurality of hair tinting products, receiving third information representative of the subject's selection of at least one hair tinting product in the plurality of hair tinting products, and providing information relating to the at least one selected hair tinting product.

In still another aspect, the method may further comprise receiving at least one of a subject's natural hair color and a subject's percentage of senescent hair.

In still another aspect, the method may further comprise determining attainable types of color result based on the subject's natural hair color, the subject's percentage of senescent hair, and the information relating to a plurality of hair tinting products.

In still another aspect, the determining attainable color results may further comprise selecting at least one of a) information relating to hair tinting products having a natural application base corresponding to the subject's natural hair color, and b) information relating to hair tinting products which satisfy at least one condition specific to at least one type of color result.

In still another aspect, wherein the desired type of color result may be selected from the attainable type of color results.

In still another aspect, wherein the first information may be received from at least one of a portable measuring device, a touch-screen display, a mouse, and a keyboard.

In still another aspect, the providing at least one type of duration may further comprise determining, based on the first information and the information relating to the hair tinting products, the product duration for each hair tinting product, and providing at least one type of product duration for selection by the subject.

In a further aspect, a method for providing hair tinting information may comprise, receiving first information representative of at least one state of a subject's hair, receiving second information representative of a duration of a hair tinting result, providing, based on at least the first information and the second information, information relating to a plurality of hair tinting products, receiving third information representative of the subject's selection of at least one hair tinting product in the plurality of hair tinting products, and providing information relating to the at least one selected hair tinting product.

In a further aspect, the receiving first information may include receiving at least one of a subject's natural hair color and a subject's percentage of senescent hair.

In a further aspect, the method may further comprise determining, based on the first information and the information relating to the hair tinting products, the product durations for each hair tinting product, and providing at least one type of product duration for selection by the subject.

In still another aspect, a method for providing hair tinting information may comprise, receiving first information representative of at least one state of a subject's hair, providing, based on at least the first information, at least one type of color result, receiving second information representative of a desired type of color result of the subject, and providing, based on at least the first information and the second information, information relating to at least one hair tinting product.

In still another aspect, the receiving second information may include receiving a desired type of color result by the user, wherein the desired type of color result is selected from the attainable type of color results.

In still another aspect, the providing information relating to the at least one selected hair tinting product may further comprise providing information relating to the hair tinting products having at least one range corresponding to the at least one desired range.

In a further aspect, a method for providing hair tinting advice may comprise receiving first information representative of a subject's percentage of senescent hair, receiving second information representative of the subject's natural hair color, determining, based on the first information, the second information, and information relating to a set of hair tinting products, attainable types of color result, and providing, based on the attainable types of color result and the set of hair tinting products, information relating to at least one hair tinting product.

In a further aspect, the method further may comprise receiving information representative of a desired type of color result chosen from the attainable types of color result, and the providing may be further based on the information representative of a desired type of color result.

In a further aspect, the information relating to at least one hair tinting product may comprise information relating to ranges of hair tinting products, and the method may further comprise receiving a subject's selection of at least one desired range, wherein the desired range provides shades attainable through one of direct application and a prior application of a bleach.

In a further aspect, the method may further comprise providing, based on the natural hair color, percentage of senescent hair, and the information relating to the set of hair tinting products, information relating to at least one duration, wherein the at least one duration corresponds to a temporal duration associated with the type of color result, and receiving third information representative of a desired duration chosen from the at least one duration.

In a further aspect, the receiving third information representative of a desired duration may include receiving one of a confirmation of a desired duration and a selection of one desired duration from a plurality of durations.

In a further aspect, the method may further comprise receiving data representative of the set of hair tinting products, wherein the data includes information representative of the ability to cover senescent hairs and the attainable type of color results.

In a further aspect, the data representative of the set of hair tinting products may include information representative of a product duration, and at least one of a natural application base, a lightening power, and a bleaching power.

In a further aspect, the method may further comprise receiving information representative of a subject's complexion, wherein the providing is further based on the subject's complexion.

In a further aspect, the method may further comprise selecting, from the set of hair tinting products, a first subset of hair tinting products corresponding to the first information, wherein the selecting is performed using a first filter.

In a further aspect, the method may further comprise selecting, from the first subset of hair tinting products, a second subset of hair tinting products corresponding to the second information, wherein the selecting is performed using a second filter.

In a further aspect, the first filter may select hair tinting products providing appropriate coverage based on the subject's percentage of senescent hair.

In a further aspect, wherein, for each determined attainable type of color result offered, the second filter may select, from the first subset, at least one of: a) hair tinting products having a natural application base corresponding to the second information, and b) hair tinting products satisfying at least one condition specific to at least one type of color result.

In a further aspect, wherein the information relating to the second subset of hair tinting products may comprise at least one duration, and wherein the method may comprise providing the at least one duration to the subject.

In a further aspect, the method may further comprise selecting, from the second subset of hair tinting products, a third subset of hair tinting products corresponding to a desired duration, wherein the selecting is performed using a third filter.

In a further aspect, the method may, further comprise acquiring an image of the subject, displaying the image of the subject on a display, and overlaying a second image on a portion of the subject's image corresponding to the subject's hair, wherein the second image is configured for a) comparing the color of the subject's hair with the at least one shade and, b) comparing the subject's percentage of senescent hair with at least one example of percentage of senescent hair.

In a further aspect, the first information and second information may be received via an input device and the providing comprises displaying the information relating to at least one product on a display device.

In a further aspect, at least the receiving of the second information may comprise receiving information obtained via a measurement device.

In a further aspect, at least one of the receiving of first information, the receiving of second information, and the providing may occur via a network.

In a further aspect, the method may further comprise prompting the subject to compare the subject's hair with one of a plurality of color swatches representing natural hair color and a plurality of color swatches representing percentages of senescent hair.

In another aspect, an apparatus for providing hair tinting information may comprise a data processor, and a memory functionally coupled to the data processor, wherein the memory stores information relating to a set hair tinting products and instructions to be executed by the data processor, for performing any of the methods described herein.

In another aspect, the apparatus may further comprise a display functionally coupled to the data processor.

In another aspect, the apparatus may further comprise an input device functionally coupled to the data processor.

In another aspect, the input device may be at least one of a keyboard, a mouse, and a measuring device.

In another aspect, the measuring device may be a portable instrument for measuring a subject's hair color.

In another aspect, the display may be a touch screen display configured to receive input commands.

In another aspect, the apparatus may further comprise a network interface functionally coupled to the data processor and a network.

In another aspect, the apparatus may further comprise a support structure which supports the data processor and the display.

In another aspect, the apparatus may further comprise an image acquisition device for capturing an image of the subject for displaying on the display.

In another aspect, said image acquisition device may comprise a camera.

It should be understood that any of the aspects described above could be combined together. In addition, it should be understood that the invention, in its broadest sense, could be practiced without one or more of the aspects described herein and also without all of the features associated with each individual aspect.

Aside from the structural and procedural arrangements set forth above, the invention could include a number of other arrangements, such as those explained hereinafter. It is to be understood that both the foregoing description and the following description are exemplary.

The accompanying drawings are incorporated in and constitute a part of this specification. The drawings illustrate some exemplary embodiments of the invention and, together with the description, serve to explain some principles of the invention. In the drawings,

FIG. 1 is an diagrammatic perspective view of an apparatus for providing hair tinting information consistent with an exemplary embodiment of the invention;

FIG. 1A is a diagram of an apparatus for measuring hair color consistent with an exemplary embodiment of the invention;

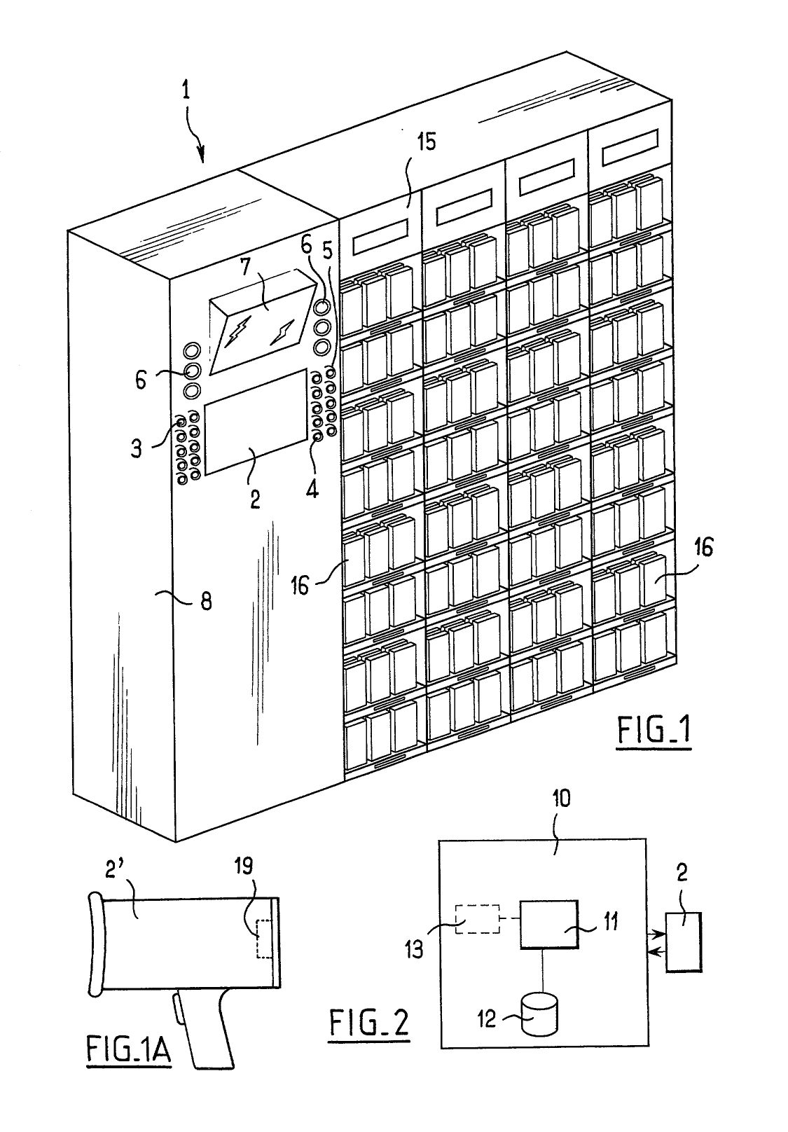

FIG. 2 is a block diagram showing the various component members of the FIG. 1 apparatus;

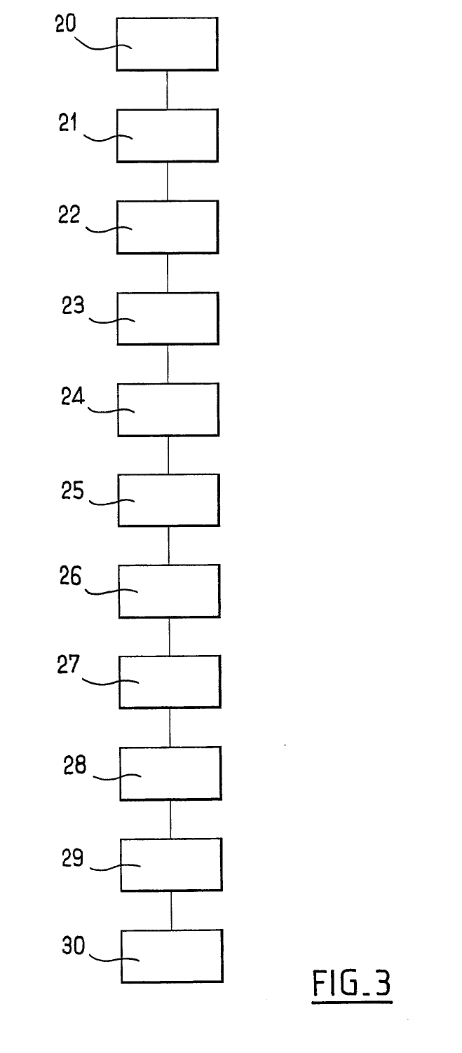

FIG. 3 is a flow diagram depicting a process for providing hair tinting information consistent with an exemplary embodiment of the invention;

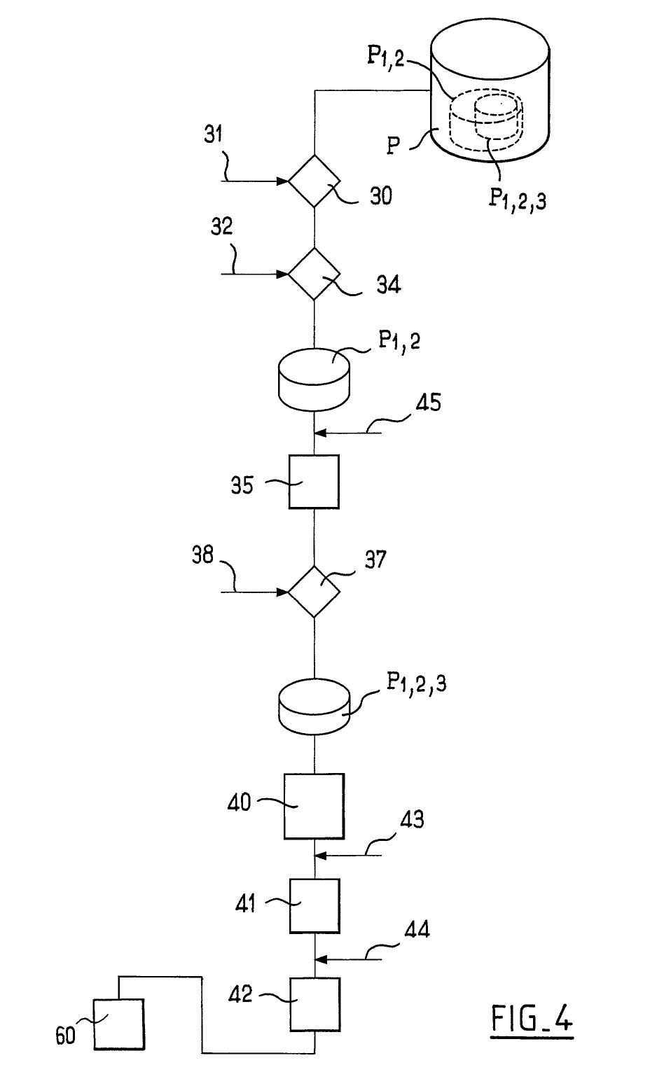

FIG. 4 is a flow diagram depicting a specific process for providing hair tinting information consistent with an exemplary embodiment of the invention;

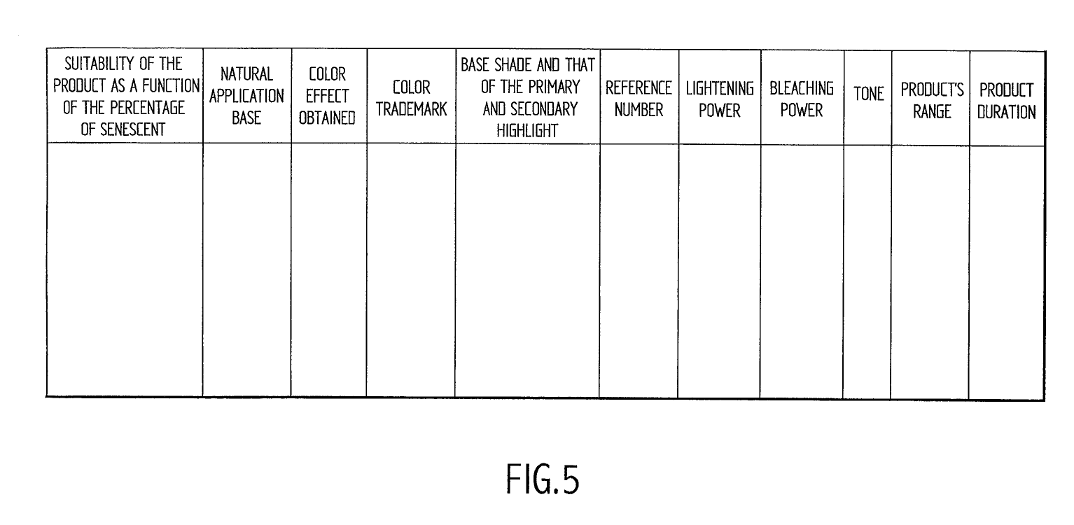

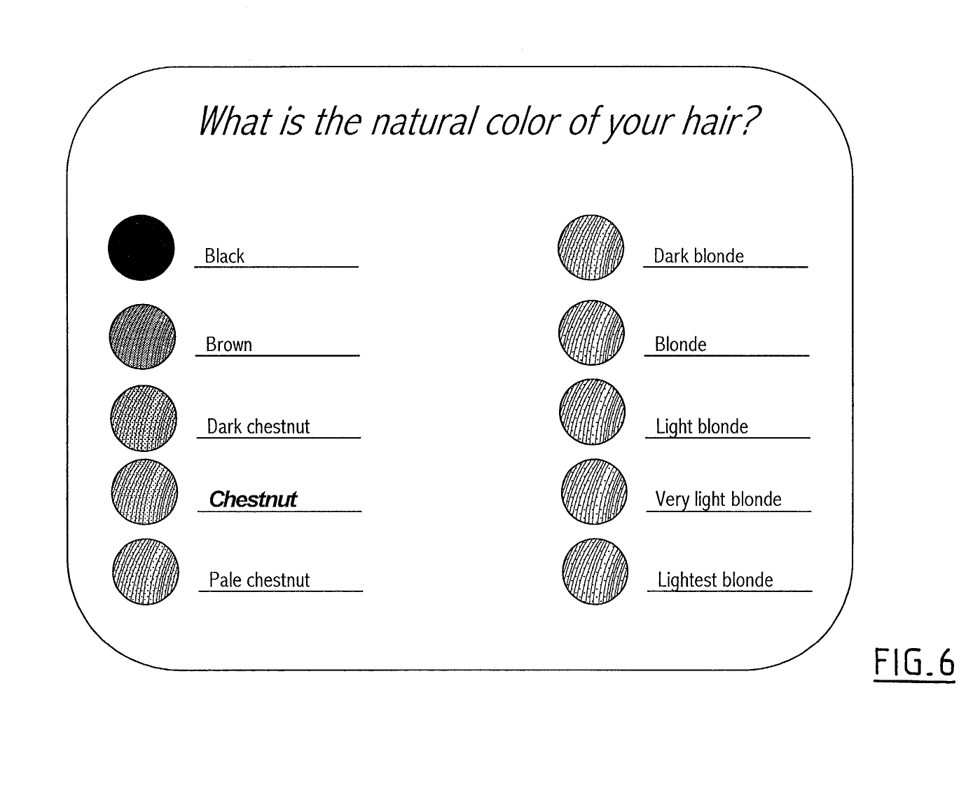

FIG. 5 is a table including information relating to hair tinting products consistent with an exemplary embodiment of the invention; and

FIGS. 6 to 15 are diagrams of information displayed on a screen consistent with an exemplary embodiment of the present invention.

Reference will now be made in detail to the exemplary embodiments of the invention, examples of which are shown in the drawings. Wherever possible, the same reference numbers are used in the drawings and the description to refer to the same like parts.

FIG. 1 depicts an apparatus 1 for providing hair tinting information consistent with an exemplary embodiment of the invention. The apparatus 1 may include a touch-sensitive screen 2, a sampler 3 of colored swatches, at least two samplers 4 and 5 of swatches of senescent hair, lighting strips 6, and a mirror 7. The assembly may also include a support 8. FIG. 1 further depicts shelves 15, on which hair tinting products 16 may be placed.

As shown in FIG. 1, the touch-sensitive screen 2 may be connected to a central unit 10 which is depicted in FIG. 2. Central unit 10 includes microprocessor 11 and data storage means 12. Microprocessor 11 may be implemented using any conventional microprocessor, such as, for example, a Pentium IV by Intel. Data storage means 12 may be implemented as, for example, a conventional hard disk, a read-only memory, a read/write memory, a DVD and/or a CD-ROM.

Alternatively (or in addition), the central unit 10 may also include interface 13 enabling the apparatus 1 to communicate with a remote server. Communication with the remote server may be implemented using conventional protocols, for example, IP protocol, in order to exchange information or to enable data to be processed remotely.

The support 8 may be configured, for example, in the form of a booth or kiosk. Support 8 may be physically located next to shelves 15 containing products 16 as listed in the apparatus 1 in a manner described in greater detail below.

Alternatively, the mirror 7 may be replaced by an image acquisition device, for example, a camera, connected to an image-overlay apparatus enabling the image observed by the camera to be displayed on a portion of the screen 2. This may allow the subject to view their own image with various hair tinting shades available, and/or aid in determining the subject's percentage of senescent hair.

For a set of listed products on offer, the data storage means 12 may contain data relating to the characteristics of the products. In one exemplary embodiment, this data may include at least the data mentioned in the table of FIG. 5, namely: suitability of the product as a function of the percentage of senescent hair; the color effect obtained, the product duration, which is the length of time the result lasts; the trademark of the shade; the base shade and the shade of the primary and secondary highlights; the reference number; the lightening power; bleaching power; tone (which may be used to make a selection depending on a subject's complexion); and the natural application base.

The information which appears for each product in the column “suitability as a function of the percentage of senescent hairs” may be, for example, the percentage of senescent hair below which the product may have sufficient covering ability to lead to a satisfactory result.

The apparatus 1 may be arranged to enable the subject to begin by stating the natural color of the subject's hair.

To do this, the apparatus 1 may proceed as shown in FIG. 3 with a first step 20 in which the screen 2 may display the page shown in FIG. 6, displaying the colors of at least ten shade numbers usually used for determining hair color, namely e.g. black, brown, dark chestnut, chestnut, light chestnut, dark blonde, blonde, light blonde, very light blonde, or lightest blonde.

Action buttons displayed on the display may coincide with each of the displayed shades.

The apparatus maybe configured to enable the subject to activate (e.g., press) an action button corresponding to the subject's own shade, and this the selected action button may correspond to the information 32 shown in FIG. 4 as described below.

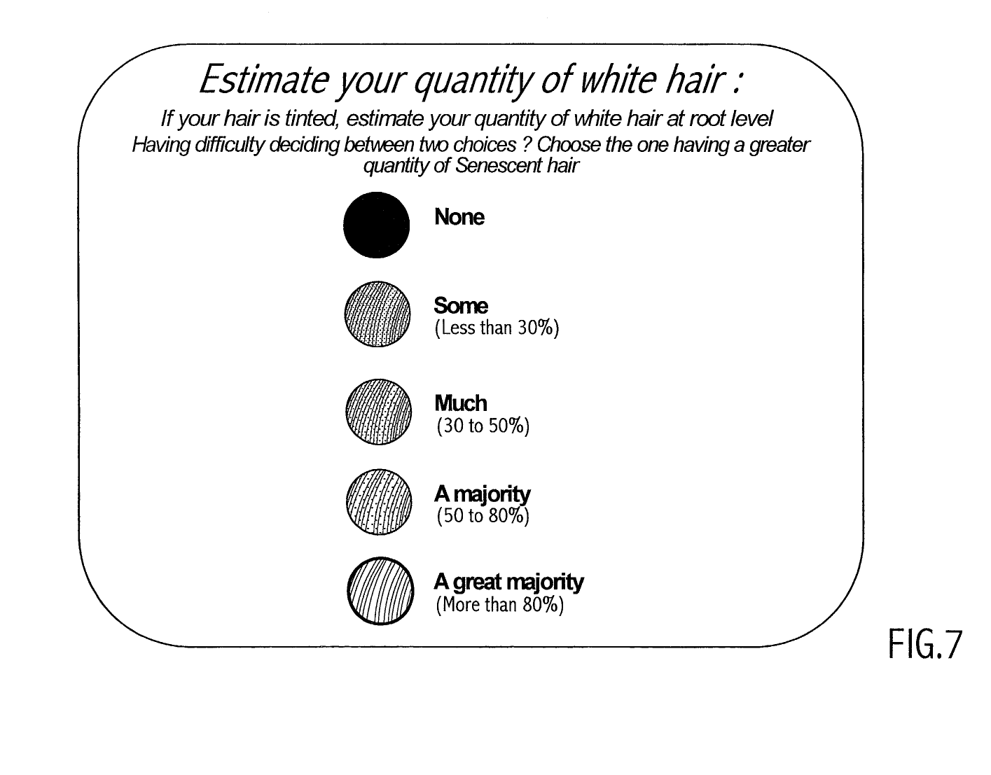

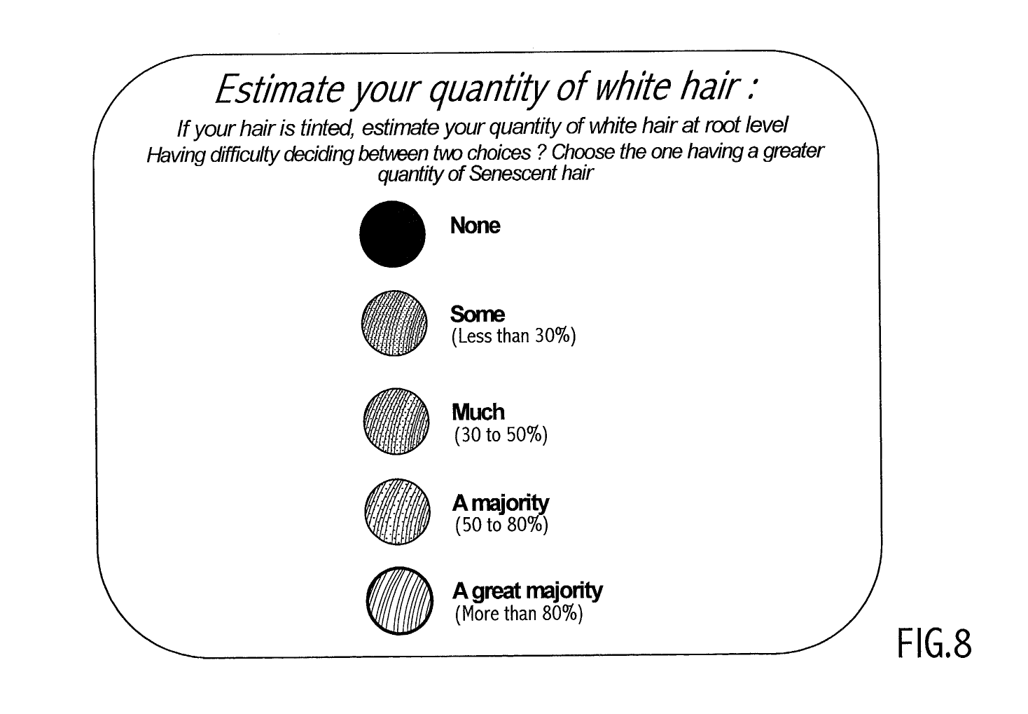

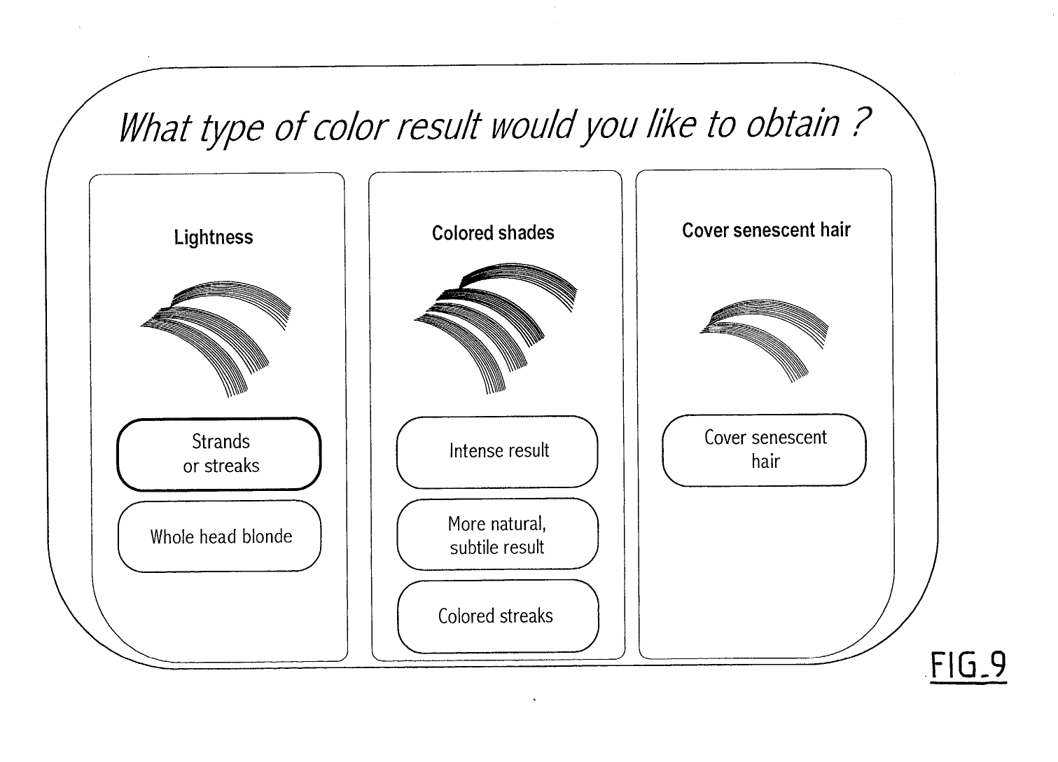

The apparatus 1 may store this natural hair color and then can proceed to step 21 where there may be either a display screen shown in FIG. 7 if the subject's hair is blonde, or a display of a screen shown in FIG. 8 if the subject's hair is darker.

The screen displays of each of FIGS. 7 and 8 may have five action buttons corresponding to senescent hair percentages that are respectively none, less than 30%, ranging from 30% to 50%, ranging from 50% to 80%, and more than 80%.

The subject may refer to the samplers 4 and 5 of senescent hair to facilitate selecting an action button corresponding to a senescent hair percentage. The selected action button may correspond to information 31 shown in FIG. 4, as explained below.

Alternatively or additionally, a device 2′ shown in FIG. 1A may be capable of measuring a subject's hair color. Device 2′ may be a hand-held or portable device which may comprise at least one detector and/or camera 19 and an appropriate means for signal processing for delivering information concerning hair color. For example, device 2′ may provide one or more numbers corresponding to the subject's base shade and/or hair highlights. The information may be directly delivered, by means of a wire or wireless connection to data processing device 11. Device 2′ may obviate the subject's manual selecting a hair color.

The device 2′ could be configured in any form capable of measuring a subject's hair color. For example, device 2′ could be configured to measure hair color as described in U.S. Pat. No. 3,736,064. More complex measurement devices, for example those comprising calorimeters and/or spectrocolorimeters, may also be used. Other examples of devices are described in WO 97/44642, WO 96/41139, and/or WO 98/11410, and could be adapted for use by any modification, if desired.

In one embodiment where the device 2′ comprises a camera 19, the camera may replace mirror 7, as previously mentioned above.

As shown in FIG. 4, apparatus 1 may include a first filter 30 for responding to information 31, representing a senescent hair percentage, and extracting, from a set of stored information relating to hair tinting products (this set shown as P in FIG. 4) those products corresponding to information 31. Apparatus 1 may also include a second filter 34 for selecting from the hair product information extracted using the first filter 30 (shown as P₁), and for each type of color result potentially on offer, those products for which: a) information 32 which represents the subject's natural hair color and the natural application bases of the listed products are compatible (when such information is available for the products); and/or b) a condition specific to the type of color result potentially on offer may be satisfied by the natural color of the hair corresponding to the information 32 and to the lightening or bleaching power of the products.

A data set P_{1,2} may be built up by applying first filter 30 and second filter 34. This data set P_{1,2} may relate to all of the products that are suitable for being offered to the subject, given the natural color of the subject's hair and the percentage of senescent hair.

The apparatus 1 may be arranged to propose the different types of color result that can be obtained through products contained in the set P_{1,2} by displaying such types of color results on the screen shown in FIG. 9.

FIG. 9 shows an example of the types of color result that may be displayed.

The type of color result “lightness, whole head blonde” maybe proposed in step 22 (and displayed as shown in FIG. 9) when a condition C+P_{E}+P_{D}≧8 is satisfied, where C is the natural color of the hair and C is expressed as a shade number lying in the range 1 to 10 from darkest to lightest (e.g., each color shown in FIG. 6 may be associated with one of 10 digits), P_{E} is the lightening power (e.g., of available tinting products) expressed as a difference of shade numbers, and P_{D} is the bleaching power (e.g., of available tinting products) expressed as a difference in shade numbers when a bleach can be used, or when the stored information associated with at least some products indicates that the product may be suitable for application on hair having the same color as the subject's hair.

The type of color result “lightness, strands or streaks” maybe proposed in step 22 when the stored data associated with at least some listed products indicates the product may be suitable for application to hair having the same color as the subject's hair.

A type of color result “colored shades, more natural, subtle” may be proposed in step 22 when products are available capable of providing shades having a shade number less than or equal to C+P_{E}+P_{D}.

Depending on the hair tinting products, the bleaching power P_{D} may be equal to no more than 4, and if the lightening power P_{E} of the product is greater than 2, then no bleaching may be possible, so the bleaching power P_{D} may equal 0.

For example, when there are a wide variety of listed products, the “colored shades, more subtle” option may be proposed by the apparatus 1.

The type of color result “colored shades, colored streaks” may be proposed in step 22 when at least one listed product may be suitable for being applied to the natural color of the subject's hair. For example, this option may always be proposed by the apparatus 1.

The type of color result “colored shades, intense result” may be proposed in step 22 when the same conditions are satisfied as for the type of color result “more natural, subtle”.

The type of color result “cover senescent hair” may be proposed in step 22 when listed products are available which can be applied to the subject's hair, given the subject's percentage of senescent hair.

In step 23, the subject selects the type of color result that may be desired by activating the corresponding action button, which corresponds to information 45 shown in FIG. 4.

In step 24, the apparatus 1 may determine the result durations that can be obtained by means of the products in the set P_{1,2}.

To do this, the apparatus 1 may have a read engine 35 arranged to read from the products in the set P_{1,2}, at least one result duration that corresponds to said products.

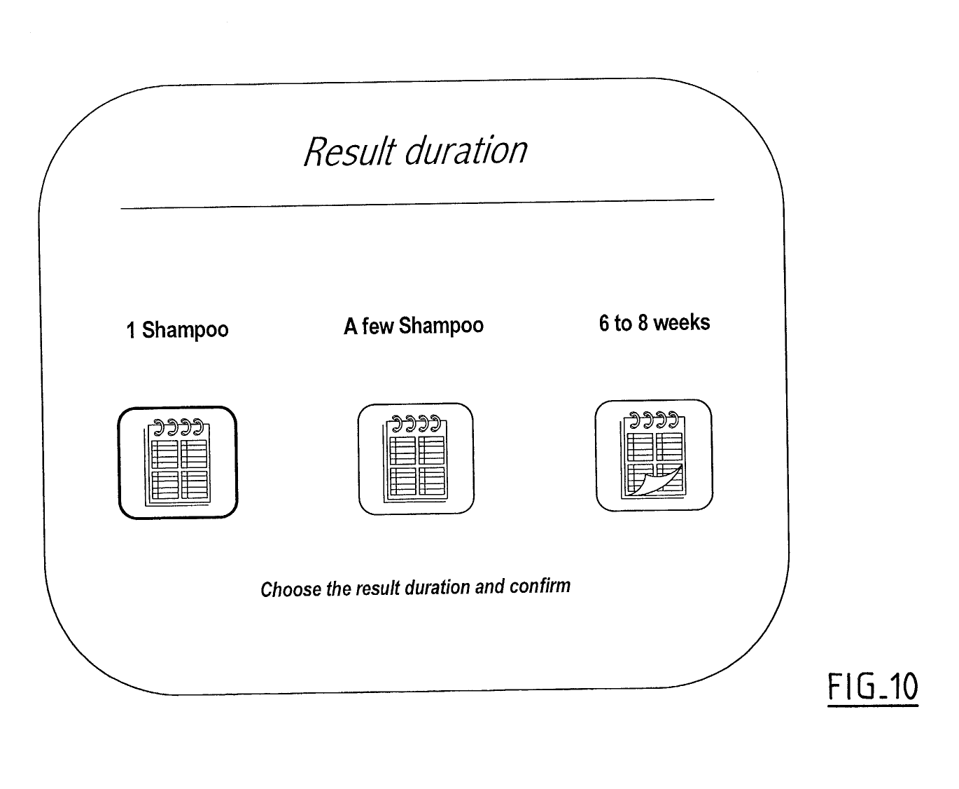

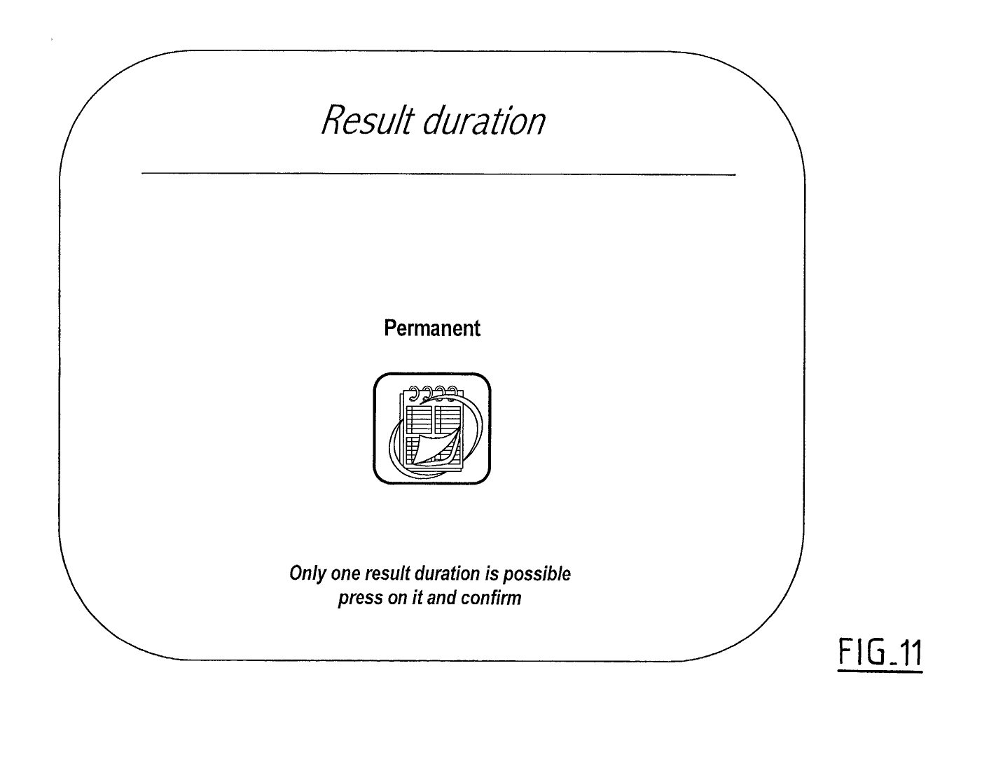

FIG. 10 shows three result durations that correspond respectively to “1 shampoo”, “a few shampoos”, and “6 to 8 weeks”, and FIG. 11 shows a display of a result whose duration is “permanent.”

In step 25, the apparatus 1 asks the subject to select a result duration, or where appropriate, to confirm the only result duration on offer, by activating on the corresponding action button, thus inputting information 38 in FIG. 4.

The apparatus may have a third filter 37 for selecting those products from the set P_{1,2} for which the result duration corresponds to the result duration selected or confirmed in step 25.

The third filter 37, which produces an information set P_{1,2,3,} may be implemented in step 26.

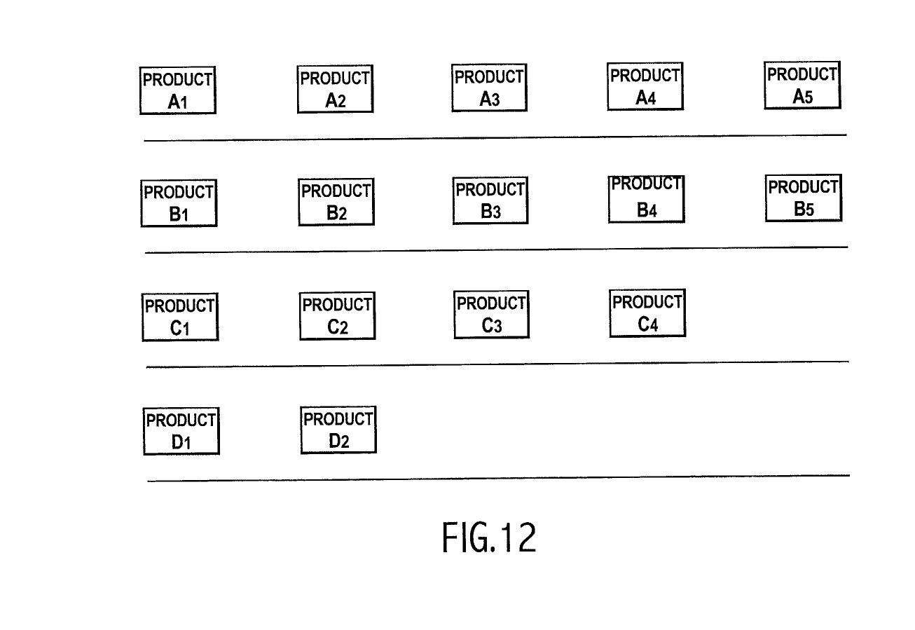

The apparatus 1 may include a read engine 40 for reading the various ranges of products that may be found in the hair tinting products represented by set P_{1,2,3.} In step 27, the apparatus displays a screen exemplified in FIG. 12. In particular, FIG. 12 shows ranges of hair tinting products as A1, . . . A₅, B₁, . . . B₅, C₁, . . . C₄, and D₁, . . . D₂, where letters A-D represent brand names and the subscripts indicate products specific to the brand name.

In step 28, the subject selects a range by activating a corresponding action button, which corresponds to information 43 shown in FIG. 4.

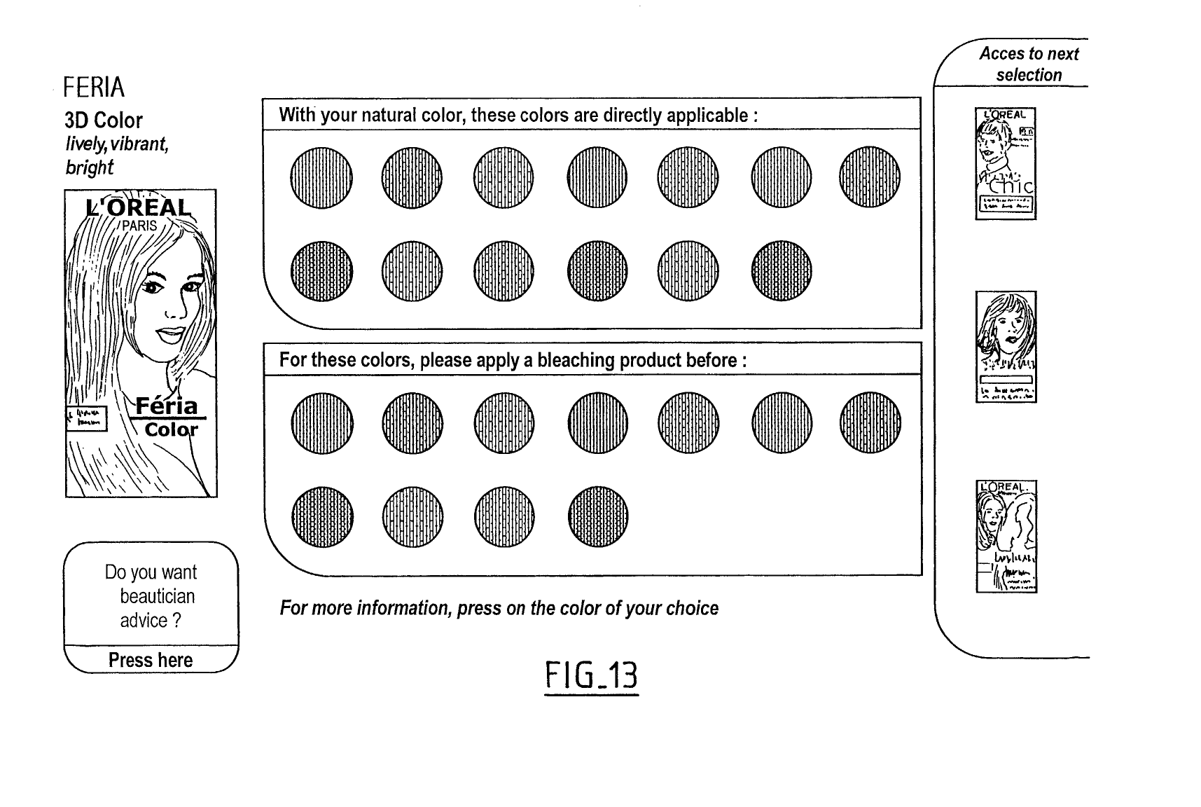

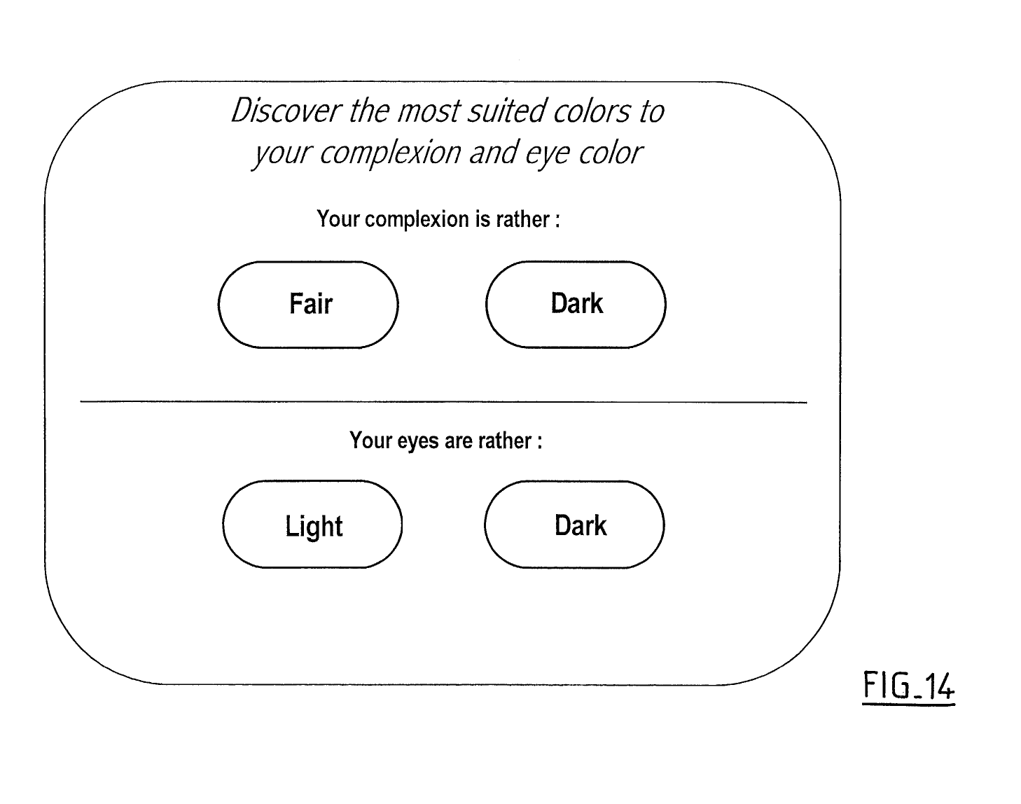

Shade display engine 41 may display the shades of the products in the selected range so they may be displayed on the screen (step 28). An exemplary display is shown in FIG. 13.

Apparatus 1 displays the shades that can be obtained by direct application of a product and those which can be obtained after applying a bleach.

Apparatus 1 may be arranged in step 29 to ask the subject to specify complexion, and in the example shown the subject may be prompted to select between “fair” or “dark” via display of a screen as shown in FIG. 14, which corresponds to the information 44 shown in FIG. 4.

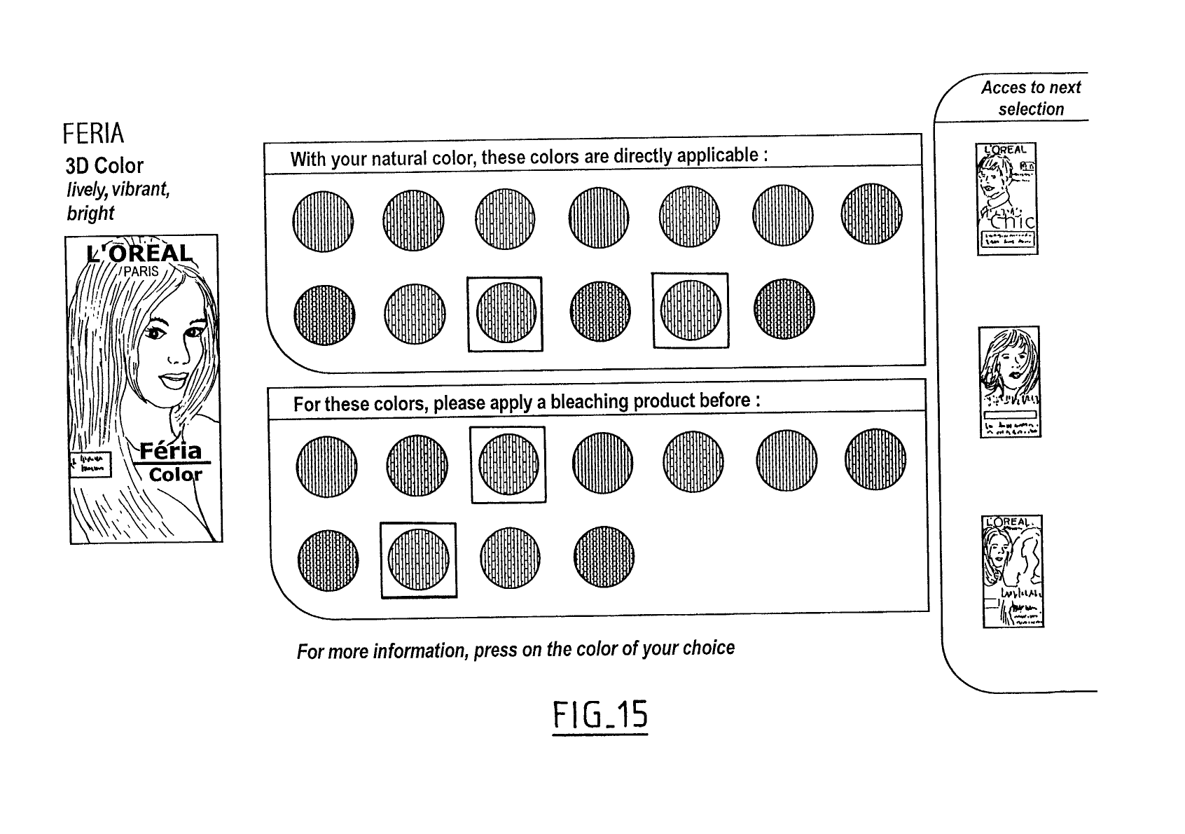

Apparatus 1 may have a read engine 42 for determining which of the displayed products are those listed as being suited to the subject's complexion (e.g., via “tone” column in the table of FIG. 5), and their may be an indication of their suitability. For example, by putting boxes around the suitable shades as shown in FIG. 15.

The following step 30 may correspond to displaying the packaging of the product selected by the subject, and possibly also giving advice on how to apply it.

Naturally, the invention is not limited to the embodiments described above. In particular, the method and apparatus could be practiced without one or more of the features associated with the embodiments described herein. For example, beautician advice step 29 can be omitted as can steps 24 and 25 for selecting or confirming a result duration.

In an alternative embodiment, apparatus 1 may also may also include an image insertion engine 60 for modifying an image of the subject, acquired by the image acquisition device, to assist the subject in providing information to apparatus 1. After an image of the subject is acquired, image insertion engine 60 may segment a portion of the image corresponding to the subject's hair. Segmentation techniques well known to those skilled in the art may be used. A second image corresponding to the segmented portion may be provided which overlays the image of the subject, and can thus modify the appearance of the subject's hair. The second image may be configured to display a shade or a percentage of senescent hair as stored by apparatus 1. This function may be used assist the subject in determining the best match for a natural hair color or a percentage of senescent hair.

Although a focus of the disclosure may be on apparatuses and server-side methods, it is nevertheless to be understood that the invention includes corresponding client side methods, software, articles of manufacture, and computer readable media, and that computer readable media can be used to store instructions for some or all of the methods described herein.

It will be apparent to those skilled in the art that various modifications and variations can be made to the structure and methodology of the present invention. Thus, it should be understood that the invention is not limited to the examples discussed in the specification. Rather, the present invention is intended to cover modifications and variations.

In the foregoing description of the embodiments, various features are grouped together in exemplary embodiments for purposes of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this description, with each claim standing on its own as a separate embodiment of the invention.

What is claimed is:

1. A method for providing hair tinting information, comprising:
receiving first information representative of at least one state of a subject's hair;
receiving second information representative of at least one desired hair tinting result of the subject;
providing information relating to a plurality of hair tinting products based on at least the first information and the second information;
receiving third information representative of the subject's selection of at least one hair tinting product in the plurality of hair tinting products; and
providing information relating to the at least one selected hair tinting product.

2. The method of claim 1, wherein the receiving first information includes receiving at least one of a subject's natural hair color and a subject's percentage of senescent hair.

3. The method of claim 2, wherein the first information is received from at least one of a portable measuring device, a touch-screen display, a mouse, and a keyboard.

4. The method of claim 2, further comprising:
providing a plurality of natural hair color examples,
wherein receiving first information comprises receiving a subject's natural hair color selection corresponding to one of the plurality of natural hair color examples which best corresponds to the subject's natural hair color; and
storing the subject's natural hair color selection.

5. The method of claim 2, further comprising:
providing a plurality of examples of senescent hair percentages,
wherein the receiving first information comprises receiving a subject's percentage of senescent hair selection corresponding to one of the plurality of examples of senescent hair percentages which best corresponds to the subject's percentage of senescent hair.

6. The method of claim 1, further comprising:
receiving product information corresponding to the plurality of hair tinting products, wherein the received product information includes at least one of suitability of covering a percentage of senescent hair, natural application base, color effect obtained, color trademark, base shade, shade of highlights, lightening power, bleaching power, tone, product range, and product duration.

7. The method of claim 6, wherein the product information is received from a local storage medium, including at least one of a hard disk and a solid state memory.

8. The method of claim 6, wherein the product information is received over a network.

9. The method of claim 8, wherein the network utilizes at least internet protocol.

10. The method of claim 6, wherein the product information is representative of products marketed under at least one brand name.

11. The method of claim 2, further comprising:
selecting from the plurality of hair tinting products, based on the subject's percentage of senescent hair, a first subset of hair tinting products; and
selecting from the first subset, based on the subject's natural hair color, a second subset of hair tinting products.

12. The method of claim 11, wherein the selecting from the first subset comprises:
selecting at least one of a) hair tinting products having a natural application base corresponding to the subject's natural hair color, and b) hair tinting products which satisfy at least one condition specific to at least one type of color result.

13. The method of claim 12, wherein the receiving second information includes receiving a desired type of color result by the user, wherein the desired type of color result is selected from attainable types of color result.

14. The method of claim 13, wherein the attainable types of color result include at least one of lightness, colored shade, and cover senescent hair.

15. The method of claim 14, wherein the lightness includes at least one of streaks and whole head.

16. The method of claim 14, wherein the colored shade includes at least one of intense result, natural result, and colored streaks result.

17. The method of claim 1, wherein the receiving second information includes receiving the desired duration of hair tinting.

18. The method of claim 17, further comprising:
determining the product durations for at least some of the hair tinting products; and
selecting hair tinting products having a product duration corresponding to the desired duration.

19. The method of claim 1, wherein the providing information relating to a plurality of hair tinting products further comprises providing ranges of hair tinting products.

20. The method of claim 19, wherein the receiving third information representative of the subject's selection further comprises receiving information associated with at least one desired range.

21. The method of claim 1, further comprising:
receiving a selection of at least one desired range of hair tinting products; and
providing information relating to the hair tinting products having at least one range corresponding to the at least one desired range.

22. The method of claim 21, wherein at least one shade is associated with the at least one range.

23. The method of claim 22, further comprising:
receiving information related to a subject's desired tone;
determining the hair tinting products within the at least one range having a tone corresponding to the desired tone; and
indicating at least one shade which corresponds to the desired tone.

24. The method of claim 1, further comprising enabling the subject to purchase at least some of the hair tinting products.

25. The method of claim 1, further comprising offering at least some of the hair tinting products for sale.

26. The method of claim 25, wherein the method is performed at least substantially entirely at a point of sale for the hair tinting products.

27. The method of claim 1, wherein the information relating to at least one hair tinting product comprises information relating to at least one of a plurality of products and a plurality of subsets of products.

28. A method for providing hair tinting information, comprising:
receiving first information representative of at least a desired type of color result of a subject;
providing at least one type of duration based at least on the first information;
receiving second information representative of a duration chosen from the at least one type of duration;
providing, based on at least the first information and the second information, information relating to a plurality of hair tinting products;
receiving third information representative of the subject's selection of at least one hair tinting product in the plurality of hair tinting products; and
providing information relating to the at least one selected hair tinting product.

29. The method of claim 28, further comprising receiving at least one of a subject's natural hair color and a subject's percentage of senescent hair.

30. The method of claim 29, further comprising:
determining attainable types of color result based on the subject's natural hair color, the subject's percentage of senescent hair, and the information relating to a plurality of hair tinting products.

31. The method of claim 30, wherein the determining attainable types of color result further comprises:
selecting at least one of a) information relating to hair tinting products having a natural application base corresponding to the subject's natural hair color, and b) information relating to hair tinting products which satisfy at least one condition specific to at least one type of color result.

32. The method of claim 30, wherein the desired type of color result is selected from the attainable types of color result.

33. The method of claim 30, wherein the attainable types of color result include at least one of lightness, colored shade, and cover senescent hair.

34. The method of claim 33, wherein the lightness includes at least one of streaks and whole head.

35. The method of claim 30, wherein the colored shade includes at least one of intense result, natural result, and colored streaks result.

36. The method of claim 28, wherein the first information is received from at least one of a portable measuring device, a touch-screen display, a mouse, and a keyboard.

37. The method of claim 29, further comprising:
providing a plurality of natural hair color examples;
receiving a subject's natural hair color selection corresponding to one of the plurality of natural hair color examples which best corresponds to the subject's natural hair color; and
storing the subject's natural hair color selection.

38. The method of claim 29, further comprising:
providing a plurality of examples of senescent hair percentages; and
receiving a subject's percentage of senescent hair selection corresponding to one of the plurality of examples of senescent hair percentages which best corresponds to the subject's percentage of senescent hair.

39. The method of claim 28, further comprising:
receiving product information corresponding to the plurality of hair tinting products, wherein the received product information includes at least one of suitability of covering a percentage of senescent hair, natural application base, color effect obtained, color trademark, base shade, shade of highlights, lightening power, bleaching power, tone, product range, and product's duration.

40. The method of claim 39, wherein the product information is received from a local storage medium, including at least one of a hard disk and a solid state memory.

41. The method of claim 39, wherein the product information is received over a network.

42. The method of claim 41, wherein the network utilizes at least internet protocol.

43. The method of claim 39, wherein the product information is representative of products marketed under at least one brand name.

44. The method of claim 28, wherein the providing at least one type of duration further comprises:
determining, based on the first information and the information relating to the hair tinting products, the product duration for each hair tinting product; and
providing at least one type of product duration for selection by the subject.

45. The method of claim 28, wherein the providing information relating to a plurality of hair tinting products further comprises providing ranges associated with the hair tinting products.

46. The method of claim 45, wherein the receiving third information representative of the subject's selection further comprises receiving information associated with at least one desired range.

47. The method of claim 28, further comprising:
receiving a selection of at least one desired range of hair tinting products; and
providing information relating to the hair tinting products having at least one range corresponding to the at least one desired range.

48. The method of claim 47, wherein at least one shade is associated with the at least one range.

49. The method of claim 48, further comprising:
receiving information related to a subject's desired tone;
determining the hair tinting products within the at least one range having a tone corresponding to the desired tone; and
indicating at least one shade which corresponds to the desired tone.

50. The method of claim 28, further comprising enabling the subject to purchase at least some of the hair tinting products.

51. The method of claim 28, further comprising offering at least some of the hair tinting products for sale.

52. The method of claim 51, wherein the method is performed at least substantially entirely at a point of sale for the hair tinting products.

53. The method of claim 28, wherein the information relating to at least one hair tinting product comprises information relating to at least one of a plurality of products and a plurality of subsets of products.

54. A method for providing hair tinting information, comprising:
receiving first information representative of at least one state of a subject's hair;
receiving second information representative of a duration of a hair tinting result;
providing, based on at least the first information and the second information, information relating to a plurality of hair tinting products;
receiving third information representative of the subject's selection of at least one hair tinting product in the plurality of hair tinting products; and
providing information relating to the at least one selected hair tinting product.

55. The method of claim 54, wherein the receiving first information includes receiving at least one of a subject's natural hair color and a subject's percentage of senescent hair.

56. The method of claim 55, wherein the first information is received from at least one of a portable measuring device, a touch-screen display, a mouse, and a keyboard.

57. The method of claim 55, further comprising:
providing a plurality of natural hair color examples,
wherein the receiving first information comprises receiving a subject's natural hair color selection corresponding to one of the plurality of natural hair color examples which best corresponds to the subject's natural hair color; and
storing the subject's natural hair color selection.

58. The method of claim 55, further comprising:
providing a plurality of examples of senescent hair percentages,
wherein the receiving first information comprises receiving a subject's percentage of senescent hair selection corresponding to one of the plurality of examples of senescent hair percentages which best corresponds to the subject's percentage of senescent hair.

59. The method of claim 54, further comprising:
receiving product information corresponding to the plurality of hair tinting products, wherein the received product information includes at least one of suitability of covering a percentage of senescent hair, natural application base, color effect obtained, color trademark, base shade, shade of highlights, lightening power, bleaching power, tone, product range, and product duration.

60. The method of claim 59, wherein the product information is received from a local storage medium, including at least one of a hard disk and a solid state memory.

61. The method of claim 59, wherein the product information is received over a network.

62. The method of claim 61, wherein the network utilizes at least internet protocol.

63. The method of claim 59, wherein the product information is representative of products marketed under at least one brand name.

64. The method of claim 54, further comprising:
determining, based on the first information and the information relating to the hair tinting products, the product durations for each hair tinting product; and
providing at least one type of product duration for selection by the subject.

65. The method of claim 54, wherein the providing information relating to a plurality of hair tinting products further comprises providing ranges of hair tinting products.

66. The method of claim 65, wherein the receiving third information representative of the subject's selection further comprises receiving information associated with at least one desired range.

67. The method of claim 54, further comprising:
receiving a selection of at least one desired range of hair tinting products; and
providing information relating to the hair tinting products having at least one range corresponding to the at least one desired range.

68. The method of claim 67, wherein at least one shade is associated with the at least one range.

69. The method of claim 68, further comprising:
receiving information related to a subject's desired tone;
determining the hair tinting products within the at least one range having a tone corresponding to the desired tone; and
indicating at least one shade which corresponds to the desired tone.

70. The method of claim 54, further comprising enabling the subject to purchase at least some of the hair tinting products.

71. The method of claim 54, further comprising offering at least some of the hair tinting products for sale.

72. The method of claim 71, wherein the method is performed at least substantially entirely at a point of sale for the hair tinting products.

73. The method of claim 54, wherein the information relating to at least one hair tinting product comprises information relating to at least one of a plurality of products and a plurality of subsets of products.

74. A method for providing hair tinting information, comprising:
receiving first information representative of at least one state of a subject's hair;
providing, based on at least the first information, at least one type of color result;
receiving second information representative of a desired type of color result of the subject; and
providing, based on at least the first information and the second information, information relating to at least one hair tinting product.

75. The method of claim 74, wherein the receiving first information includes receiving at least one of a subject's natural hair color and a subject's percentage of senescent hair.

76. The method of claim 75, wherein the first information is received from at least one of a portable measuring device, a touch-screen display, a mouse, and a keyboard.

77. The method of claim 75, further comprising:
providing a plurality of natural hair color examples,
wherein the receiving first information comprises receiving a subject's natural hair color selection corresponding to one of the plurality of natural hair color examples which best corresponds to the subject's natural hair color; and
storing the subject's natural hair color selection.

78. The method of claim 75, further comprising:
providing a plurality of examples of senescent hair percentages; and
receiving a subject's percentage of senescent hair selection corresponding to one of the plurality of percentages of senescent hair examples which best corresponds to the subject's percentage of senescent hair.

79. The method of claim 74, further comprising:
receiving product information corresponding to a plurality of hair tinting products, wherein the received product information includes at least one of suitability of covering a percentage of senescent hair, natural application base, color effect obtained, color trademark, base shade, shade of highlights, lightening power, bleaching power, tone, product range, and product duration.

80. The method of claim 79, wherein the product information is received from a local storage medium, including at least one of a hard disk and a solid state memory.

81. The method of claim 79, wherein the product information is received over a network.

82. The method of claim 81, wherein the network utilizes at least internet protocol.

83. The method of claim 79, wherein the product information is representative of products marketed under at least one brand name.

84. The method of claim 75, further comprising:
selecting from a plurality of hair tinting products, based on the subject's percentage of senescent hair, a first subset of hair tinting products; and
selecting from the first subset, based on the subject's natural hair color, a second subset of hair tinting products.

85. The method of claim 84 wherein the selecting from the first subset comprises:
selecting at least one of a) hair tinting products having a natural application base corresponding to the subject's natural hair color, and b) hair tinting products which satisfy at least one condition specific to at least one type of color result.

86. The method of claim 85, wherein the receiving second information includes receiving a desired type of color result by the user, wherein the desired type of color result is selected from attainable types of color result.

87. The method of claim 86, wherein the attainable types of color result include at least one of lightness, colored shade, and cover senescent hair.

88. The method of claim 87, wherein the lightness includes at least one of streaks and whole head.

89. The method of claim 87, wherein the colored shade includes at least one of intense result, natural result, and colored streaks result.

90. The method of claim 74, wherein the providing information relating to at least one hair tinting product further comprises determining ranges associated with hair tinting products.

91. The method of claim 90, further comprising receiving information associated with at least one desired range.

92. The method of claim 91, wherein the providing information relating to the at least one hair tinting product further comprises providing information relating to the hair tinting products having at least one range corresponding to the at least one desired range.

93. The method of claim 92, wherein at least one shade is associated with the at least one range.

94. The method of claim 93, further comprising:
receiving information related to a subject's desired tone;
determining the hair tinting products within the at least one range having a tone corresponding to the desired tone; and
indicating at least one shade which corresponds to the desired tone.

95. The method of claim 74, further comprising enabling the subject to purchase at least some hair tinting products.

96. The method of claim 74, further comprising offering at least some hair tinting products for sale.

97. The method of claim 96, wherein the method is performed at least substantially entirely at a point of sale for the hair tinting products.

98. The method of claim 74, wherein the information relating to at least one hair tinting product comprises information relating to at least one of a plurality of products and a plurality of subsets of products.

99. A method for providing hair tinting advice, comprising:
receiving first information representative of a subject's percentage of senescent hair;
receiving second information representative of the subject's natural hair color;
determining, based on the first information, the second information, and information relating to a set of hair tinting products, attainable types of color result; and
providing, based on the attainable types of color result and the set of hair tinting products, information relating to at least one hair tinting product.

100. The method of claim 99, wherein the method further comprises receiving information representative of a desired type of color result chosen from the attainable types of color result, and wherein the providing is further based on the information representative of a desired type of color result.

101. The method of claim 100, wherein the information relating to at least one hair tinting product comprises information relating to ranges of hair tinting products, and wherein the method further comprises receiving a subject's selection of at least one desired range, wherein the desired range provides shades attainable through one of direct application and a prior application of a bleach.

102. The method of claim 99, further comprising:
providing, based on the natural hair color, percentage of senescent hair, and the information relating to the set of hair tinting products, information relating to at least one duration, wherein the at least one duration corresponds to a temporal duration associated with the type of color result; and
receiving third information representative of a desired duration chosen from the at least one duration.

103. The method of claim 102, wherein the receiving third information representative of a desired duration includes receiving one of a confirmation of a desired duration and a selection of one desired duration from a plurality of durations.

104. The method of claim 99, further comprising receiving data representative of the set of hair tinting products, wherein the data includes information representative of the ability to cover senescent hair and the attainable types of color result.

105. The method of claim 104, wherein the data representative of the set of hair tinting products includes information representative of a product duration, and at least one of a natural application base, a lightening power, and a bleaching power.

106. The method of claim 99, further comprising receiving information representative of a subject's complexion, wherein the providing is further based on the information representative of the subject's complexion.

107. The method of claim 99, further comprising selecting, from the set of hair tinting products, a first subset of hair tinting products corresponding to the first information, wherein the selecting is performed using a first filter.

108. The method of claim 107, further comprising selecting, from the first subset of hair tinting products, a second subset of hair tinting products corresponding to the second information, wherein the selecting is performed using a second filter.

109. The method of claim 107, wherein the first filter selects hair tinting products providing appropriate coverage based on the subject's percentage of senescent hair.

110. The method of claim 108, wherein, for each determined attainable type of color result offered, the second filter selects, from the first subset, at least one of: a) information relating to hair tinting products having a natural application base corresponding to the second information, and b) information relating to hair tinting products satisfying at least one condition specific to at least one type of color result.

111. The method of claim 99, further comprising providing an attainable type of color result corresponding to blonde hair for the whole head when a condition C+Pe+Pd≧8 is satisfied, wherein C represents the subject's natural hair color, Pe represents a lightening power, and Pd represents a bleaching power.

112. The method of claim 99, further comprising providing an attainable type of color result corresponding to blonde hair over only a portion of the hair when the hair tinting product is suitable for application to hair having the same color as the subject's natural hair color.

113. The method of claim 99, further comprising providing an attainable type of color result corresponding to hair that is not blonde and having a color that is different from the at least a portion of the subject's natural hair color when the hair tinting product is suitable for application to hair having the same color as the subject's natural hair color.

114. The method of claim 99, further comprising providing an attainable type of color result corresponding to shading senescent hair, when the hair tinting product is suitable for application to hair having the same color as the subject's natural hair color.

115. The method of claim 108, wherein the information relating to the second subset of hair tinting products comprises at least one duration, and wherein the method comprises providing the at least one duration to the subject.

116. The method of claim 115, further comprising selecting, from the second subset of hair tinting products, a third subset of hair tinting products corresponding to a desired duration, wherein the selecting is performed using a third filter.

117. The method of claim 116 wherein the set of hair tinting products includes ranges of hair tinting products, and wherein each range includes at least one hair tinting product.

118. The method of claim 117, further comprising reading, from at least one of the second subset and the third subset, ranges associated with the information related to the hair tinting products.

119. The method of claim 101, further comprising providing shades of the hair tinting products corresponding to the desired range selected by the subject.

120. The method of claim 116, further comprising:
reading, from at least one of the second subset and the third subset, at least one shade associated with the information relating to the at least one hair tinting product, wherein the shade is suitable for the subject's complexion.

121. The method of claim 99, further comprising:
acquiring an image of the subject;
displaying the image of the subject on a display; and
overlaying a second image on a portion of the subject's image corresponding to the subject's hair, wherein the second image is configured for a) comparing the color of the subject's hair with the at least one shade and, b) comparing the subject's percentage of senescent hair with at least one example of percentage of senescent hair.

122. The method of claim 99, further comprising enabling the subject to purchase at least some of the hair tinting products.

123. The method of claim 99, further comprising offering at least some of the hair tinting products for sale.

124. The method of claim 123, wherein the method is performed at least substantially entirely at a point of sale for the hair tinting products.

125. The method of claim 99, wherein the information relating to at least one hair tinting product comprises information relating to at least one of a plurality of products and a plurality of subsets of products.

126. The method of claim 99, wherein the first information and second information are received via an input device and the providing comprises displaying the information relating to at least one product on a display device.

127. The method of claim 99, wherein at least the receiving of the second information comprises receiving information obtained via a measurement device.

128. The method of claim 99, wherein at least one of the receiving of first information, the receiving of second information, and the providing occurs via a network.

129. The method of claim 99, further comprising prompting the subject to compare the subject's hair with one of a plurality of color swatches representing natural hair color and a plurality of color swatches representing percentages of senescent hair.

130. An apparatus for providing hair tinting information, comprising
a data processor; and
a memory functionally coupled to the data processor, wherein the memory stores information relating to a set hair tinting products and instructions to be executed by the data processor, for:
receiving first information representative of a subject's percentage of senescent hair;
receiving second information representative of the subject's natural hair color;
determining, based on the first information, the second information, and information relating to a set of hair tinting products, attainable types of color results; and
providing, based on the attainable types of color results and the set of hair tinting products, information relating to at least one hair tinting product.

131. The apparatus of claim 130, further comprising a display functionally coupled to the data processor.

132. The apparatus of claim 130, further comprising an input device functionally coupled to the data processor.

133. The apparatus of claim 130, wherein the input device is at least one of a keyboard, a mouse, and a measuring device.

134. The apparatus of claim 133, wherein the measuring device is a portable instrument for measuring a subject's hair color.

135. The apparatus of claim 131, wherein the display is a touch screen display configured to receive input commands.

136. The apparatus of claim 130, further comprising a network interface functionally coupled to the data processor and a network.

137. The apparatus of claim 131, further comprising a support structure which supports the data processor and the display.

138. The apparatus of claim 130, further comprising additional instructions stored in memory, to be executed by the data processor, for receiving information representative of a desired color result chosen from the attainable types of color results, and wherein the providing is further based on the information representative of a desired color result.

139. The apparatus of claim 130, wherein the information relating to at least one hair tinting product comprises information relating to ranges of hair tinting products, and wherein the apparatus further comprises additional instructions stored in memory executed by the data processor, for receiving a subject's selection of at least one desired range, wherein the desired range provides shades attainable through one of direct application and a prior application of a bleach.

140. The apparatus of claim 130, further comprising additional instructions to be executed by the data processor for:
providing, based on the natural hair color, percentage of senescent hair, and the information relating to the set of hair tinting products, information relating to at least one duration, wherein the at least one duration corresponds to a temporal duration of the color result; and
receiving third information representative of a desired duration chosen from the at least one duration.

141. The apparatus of claim 140, wherein the receiving third information representative of a desired duration includes receiving one of a confirmation of a desired duration and a selection of one desired duration from a plurality of durations.

142. The apparatus of claim 130, further comprising additional instructions to be executed by the data processor for receiving data representative of the set of hair tinting products, wherein the data includes information representative of the ability to cover senescent hairs and the attainable types of color result.

143. The apparatus of claim 142, wherein the data representative of the set of hair tinting products includes information representative of a product duration, and at least one of a natural application base, a lightening power, and a bleaching power.

144. The apparatus of claim 130, further comprising additional instructions to be executed by the data processor for receiving information representative of a subject's complexion, wherein the providing is further based on the information representative of the subject's complexion.

145. The apparatus of claim 130, further comprising additional instructions to be executed by the data processor for selecting, from the set of hair tinting products, a first subset of hair tinting products corresponding to the first information, wherein the selecting is performed using a first filter.

146. The apparatus of claim 145, further comprising additional instructions to be executed by the data processor for selecting, from the first subset of hair tinting products, a second subset of hair tinting products corresponding to the second information, wherein the selecting is performed using a second filter.

147. The apparatus of claim 145, wherein the first filter selects hair tinting products providing appropriate coverage based on the subject's percentage of senescent hair.

148. The apparatus of claim 146, wherein, for each determined attainable type of color result offered, the second filter selects, from the first subset, at least one of: a) information relating to hair tinting products having a natural application base corresponding to the second information, and b) information relating to hair tinting products satisfying a condition specific to types of color result.

149. The apparatus of claim 130, further comprising providing an attainable type of color result corresponding to blonde hair for the whole head when a condition C+Pe+Pd≧8 is satisfied, wherein C represents the subject's natural hair color, Pe represents a lightening power, and Pd represents a bleaching power.

150. The apparatus of claim 130, further comprising additional instructions for execution on the data processor for providing an attainable type of color result corresponding to blonde hair over only a portion of the hair when the hair tinting product is suitable for application to hair having the same color as the subject's natural hair color.

151. The apparatus of claim 130, further comprising additional instructions for execution on the data processor for providing an attainable type of color result corresponding to hair that is not blonde and having a color that is different from the at least a portion of the subject's natural hair color when the hair tinting product is suitable for application to hair having the same color as the subject's natural hair color.

152. The apparatus of claim 130, further comprising providing an attainable type of color result corresponding to shading senescent hair, when the hair tinting product is suitable for application to hair having the same color as the subject's natural hair color.

153. The apparatus of claim 146, wherein the information relating to the second subset of hair tinting products comprises at least one duration, and wherein the method comprises providing the at least one duration to the subject.

154. The apparatus of claim 153, further comprising additional instructions for execution on the data processor for selecting, from the second subset of hair tinting products, a third subset of hair tinting products corresponding to a desired duration, wherein the selecting is performed using a third filter.

155. The apparatus of claim 154 wherein the set of hair tinting products includes ranges of hair tinting products, and wherein each range includes at least one hair tinting product.

156. The apparatus of claim 155, further comprising additional instructions for execution on the data processor for reading, from at least one of the second subset and the third subset, ranges associated with the hair tinting products.

157. The apparatus of claim 139, further comprising additional instructions for execution on the data processor for providing shades of the hair tinting products corresponding to the desired range selected by the subject.

158. The apparatus of claim 154, further comprising additional instructions for execution on the data processor for:
reading, from at least one of the second subset and the third subset, at least one shade associated with the information relating to the at least one hair tinting product, wherein the shade is suitable for the subject's complexion.

159. The apparatus of claim 132, further comprising:
an image acquisition device for capturing an image of the subject for displaying on the display.

160. The apparatus of claim 159, wherein said image acquisition device comprises a camera.

161. The apparatus of claim 159, further comprising additional instructions for execution on the data processor for overlaying a second image on a portion of the subject's image corresponding to the subject's hair, wherein the second image is configured for a) comparing the color of the subject's hair with the at least one shade and, b) comparing the subject's percentage of senescent hair with at least one example of percentage of senescent hair.

162. The apparatus of claim 130, further comprising additional instructions for execution on the data processor for enabling the subject to purchase at least some of the hair tinting products.

163. The apparatus of claim 130, further comprising additional instructions for execution on the data processor for offering at least some of the hair tinting products for sale.

164. The apparatus of claim 130, wherein the apparatus operates at least substantially entirely at a point of sale for the hair tinting products.

165. The apparatus of claim 130, wherein the information relating to at least one hair tinting product comprises information relating to at least one of a plurality of products and a plurality of subsets of products.

166. The apparatus of claim 130, wherein the first information and second information are received via an input device and the providing comprises displaying the information relating to at least one product on a display device.

167. The method of claim 130, wherein at least the receiving of the second information comprises receiving information obtained via a measurement device.

168. The apparatus of claim 130, wherein at least one of the receiving of first information, the receiving of second information, and the providing occurs via a network.

169. The apparatus of claim 130, further comprising additional instructions for execution on the data processor for prompting the subject to compare the subject's hair with one of a plurality of color swatches representing natural hair color and a plurality of color swatches representing percentages of senescent hair.

170. The apparatus of claim 130, wherein the apparatus operates substantially as a stand-alone unit without being connected to a computer server.

171. The apparatus of claim 130, further comprising a mirror.

172. The apparatus of claim 130, further comprising at least one swatch sampler.

173. The apparatus of claim 172, wherein the at least one swatch sampler includes a sampler of colored swatches comprising shade colors of black, brown, dark chestnut, chestnut, pale chestnut, dark blonde, blonde, light blonde, very light blonde, and lightest blonde.

174. The apparatus of claim 172, further comprising at least one swatch sampler for estimating the quantity of senescent hair.

175. The apparatus of claim 174, wherein the at least one swatch sampler further includes two sets of samplers enabling the quantity of senescent hair to be evaluated, one corresponding to a subject having fair hair and one corresponding to a subject having dark hair.

176. The apparatus of claim 174, wherein the at least one swatch sampler for estimating the quantity of senescent hair is comprised of at least five swatches, corresponding to 0%, less than 30%, 30% to 50%, 50% to 80%, and greater than 80%.

177. The apparatus of claim 130, wherein the apparatus is adjacent to at least one set of shelves suitable for hair tinting products.

178. The apparatus of claim 130, further comprising a lighting means for lighting a subject's hair.

179. An apparatus for providing hair tinting information, comprising
a data processor; and
a memory functionally coupled to the data processor, wherein the memory stores information relating to a set hair tinting products and instructions to be executed by the data processor, for performing the method of claim 1.

180. The apparatus of claim 179, further comprising a display functionally coupled to the data processor.

181. The apparatus of claim 179, further comprising an input device functionally coupled to the data processor.

182. The apparatus of claim 181, wherein the input device is at least one of a keyboard, a mouse, and a measuring device.

183. The apparatus of claim 182, wherein the measuring device is a portable instrument for measuring a subject's hair color.

184. The apparatus of claim 180, wherein the display is a touch screen display configured to receive input commands.

185. The apparatus of claim 179, further comprising a network interface functionally coupled to the data processor and a network.

186. The apparatus of claim 180, further comprising a support structure which supports the data processor and the display.

187. The apparatus of claim 180, further comprising:
an image acquisition device for capturing an image of the subject for displaying on the display.

188. The apparatus of claim 187, wherein said image acquisition device comprises a camera.

189. The apparatus of claim 179, wherein the apparatus operates at least substantially entirely at a point of sale for the hair tinting products.

190. The apparatus of claim 179, wherein the apparatus operates substantially as a stand-alone unit without being connected to a computer server.

191. The apparatus of claim 179, further comprising a mirror.

192. The apparatus of claim 179, further comprising at least one swatch sampler.

193. The apparatus of claim 192, wherein the at least one swatch sampler includes a sampler of colored swatches comprising shade colors of black, brown, dark chestnut, chestnut, pale chestnut, dark blonde, blonde, light blonde, very light blonde, and lightest blonde.

194. The apparatus of claim 192, further comprising at least one swatch sampler for estimating the quantity of senescent hair.

195. The apparatus of claim 194, wherein the at least one swatch sampler further includes two sets of samplers enabling the quantity of senescent hair to be evaluated, one corresponding to a subject having fair hair and one corresponding to a subject having dark hair.

196. The apparatus of claim 194, wherein the at least one swatch sampler for estimating the quantity of senescent hair is comprised of at least five swatches, corresponding to 0%, less than 30%, 30% to 50%, 50% to 80%, and greater than 80%.

197. The apparatus of claim 179, wherein the apparatus is adjacent to at least one set of shelves suitable for hair tinting products.

198. The apparatus of claim 179, further comprising a lighting means for lighting a subject's hair.

199. An apparatus for providing hair tinting information, comprising
a data processor; and
a memory functionally coupled to the data processor, wherein the memory stores information relating to a set hair tinting products and instructions to be executed by the data processor, for performing the method of claim 28.

200. The apparatus of claim 199, further comprising a display functionally coupled to the data processor.

201. The apparatus of claim 199, further comprising an input device functionally coupled to the data processor.

202. The apparatus of claim 201, wherein the input device is at least one of a keyboard, a mouse, and a measuring device.

203. The apparatus of claim 202, wherein the measuring device is a portable instrument for measuring a subject's hair color.

204. The apparatus of claim 200, wherein the display is a touch screen display configured to receive input commands.

205. The apparatus of claim 199, further comprising a network interface functionally coupled to the data processor and a network.

206. The apparatus of claim 200, further comprising a support structure which supports the data processor and the display.

207. The apparatus of claim 200, further comprising:
an image acquisition device for capturing an image of the subject for displaying on the display.

208. The apparatus of claim 207, wherein said image acquisition device comprises a camera.

209. The apparatus of claim 199, wherein the apparatus operates at least substantially entirely at a point of sale for the hair tinting products.

210. The apparatus of claim 199, wherein the apparatus operates substantially as a stand-alone unit without being connected to a computer server.

211. The apparatus of claim 199, further comprising a mirror.

212. The apparatus of claim 199, further comprising at least one swatch sampler.

213. The apparatus of claim 212, wherein the at least one swatch sampler includes a sampler of colored swatches comprising shade colors of black, brown, dark chestnut, chestnut, pale chestnut, dark blonde, blonde, light blonde, very light blonde, and lightest blonde.

214. The apparatus of claim 212, further comprising at least one swatch sampler for estimating the quantity of senescent hair.

215. The apparatus of claim 214, wherein the at least one swatch sampler further includes two sets of samplers enabling the quantity of senescent hair to be evaluated, one corresponding to a subject having fair hair and one corresponding to a subject having dark hair.

216. The apparatus of claim 214, wherein the at least one swatch sampler for estimating the quantity of senescent hair is comprised of at least five swatches, corresponding to 0%, less than 30%, 30% to 50%, 50% to 80%, and greater than 80%.

217. The apparatus of claim 199, wherein the apparatus is adjacent to at least one set of shelves suitable for hair tinting products.

218. The apparatus of claim 199, further comprising a lighting means for lighting a subject's hair.

219. An apparatus for providing hair tinting information, comprising
a data processor; and
a memory functionally coupled to the data processor, wherein the memory stores information relating to a set hair tinting products and instructions to be executed by the data processor, for performing the method of claim 54.

220. The apparatus of claim 219, further comprising a display functionally coupled to the data processor.

221. The apparatus of claim 219, further comprising an input device functionally coupled to the data processor.

222. The apparatus of claim 221, wherein the input device is at least one of a keyboard, a mouse, and a measuring device.

223. The apparatus of claim 222, wherein the measuring device is a portable instrument for measuring a subject's hair color.

224. The apparatus of claim 220, wherein the display is a touch screen display configured to receive input commands.

225. The apparatus of claim 219, further comprising a network interface functionally coupled to the data processor and a network.

226. The apparatus of claim 220, further comprising a support structure which supports the data processor and the display.

227. The apparatus of claim 220, further comprising:
an image acquisition device for capturing an image of the subject for displaying on the display.

228. The apparatus of claim 227, wherein said image acquisition device comprises a camera.

229. The apparatus of claim 219, wherein the apparatus operates at least substantially entirely at a point of sale for the hair tinting products.

230. The apparatus of claim 219, wherein the apparatus operates substantially as a stand-alone unit without being connected to a computer server.

231. The apparatus of claim 219, further comprising a mirror.

232. The apparatus of claim 219, further comprising at least one swatch sampler.

233. The apparatus of claim 232, wherein the at least one swatch sampler includes a sampler of colored swatches comprising shade colors of black, brown, dark chestnut, chestnut, pale chestnut, dark blonde, blonde, light blonde, very light blonde, and lightest blonde.

234. The apparatus of claim 232, further comprising at least one swatch sampler for estimating the quantity of senescent hair.

235. The apparatus of claim 234, wherein the at least one swatch sampler further includes two sets of samplers enabling the quantity of senescent hair to be evaluated, one corresponding to a subject having fair hair and one corresponding to a subject having dark hair.

236. The apparatus of claim 234, wherein the at least one swatch sampler for estimating the quantity of senescent hair is comprised of at least five swatches, corresponding to 0%, less than 30%, 30% to 50%, 50% to 80%, and greater than 80%.

237. The apparatus of claim 219, wherein the apparatus is adjacent to at least one set of shelves suitable for hair tinting products.

238. The apparatus of claim 219, further comprising a lighting means for lighting a subject's hair.

239. An apparatus for providing hair tinting information, comprising
a data processor; and
a memory functionally coupled to the data processor, wherein the memory stores information relating to a set hair tinting products and instructions to be executed by the data processor, for performing the method of claim 74.

240. The apparatus of claim 239, further comprising a display functionally coupled to the data processor.

241. The apparatus of claim 239, further comprising an input device functionally coupled to the data processor.

242. The apparatus of claim 241, wherein the input device is at least one of a keyboard, a mouse, and a measuring device.

243. The apparatus of claim 242, wherein the measuring device is a portable instrument for measuring a subject's hair color.

244. The apparatus of claim 240, wherein the display is a touch screen display configured to receive input commands.

245. The apparatus of claim 239, further comprising a network interface functionally coupled to the data processor and a network.

246. The apparatus of claim 240, further comprising a support structure which supports the data processor and the display.

247. The apparatus of claim 240, further comprising:
an image acquisition device for capturing an image of the subject for displaying on the display.

248. The apparatus of claim 247, wherein said image acquisition device comprises a camera.

249. The apparatus of claim 239, wherein the apparatus operates at least substantially entirely at a point of sale for the hair tinting products.

250. The apparatus of claim 239, wherein the apparatus operates substantially as a stand-alone unit without being connected to a computer server.

251. The apparatus of claim 239, further comprising a mirror.

252. The apparatus of claim 239, further comprising at least one swatch sampler.

253. The apparatus of claim 252, wherein the at least one swatch sampler includes a sampler of colored swatches comprising shade colors of black, brown, dark chestnut, chestnut, pale chestnut, dark blonde, blonde, light blonde, very light blonde, and lightest blonde.

254. The apparatus of claim 252, further comprising at least one swatch sampler for estimating the quantity of senescent hair.

255. The apparatus of claim 254, wherein the at least one swatch sampler further includes two sets of samplers enabling the quantity of senescent hair to be evaluated, one corresponding to a subject having fair hair and one corresponding to a subject having dark hair.

256. The apparatus of claim 254, wherein the at least one swatch sampler for estimating the quantity of senescent hair is comprised of at least five swatches, corresponding to 0%, less than 30%, 30% to 50%, 50% to 80%, and greater than 80%.

257. The apparatus of claim 239, wherein the apparatus is adjacent to at least one set of shelves suitable for hair tinting products.

258. The apparatus of claim 239, further comprising a lighting means for lighting a subject's hair.
